(19) United States
(12) Patent Application Publication
(10) Pub. No.: US 2025/0002869 A1
(43) Pub. Date: Jan. 2, 2025

(54) Burkholderia pseudomallei (Bp) phage vB BpP HN01 and use thereof

(71) Applicant: Hainan Medical University, Haikou City (CN)

(72) Inventors: Qianfeng XIA, Haikou City (CN); Yanshuang WANG, Haikou City (CN); Shen TIAN, Haikou City (CN); Nini LUO, Haikou City (CN); Xuemiao LI, Haikou City (CN)

(21) Appl. No.: 18/345,524

(22) Filed: Jun. 30, 2023

(51) Int. Cl. C12N 7/00; A61K 35/76

(52) U.S. Cl. CPC C12N 7/00; A61K 35/76; C12N 2795/10033

(57) ABSTRACT

A purified phage having a specific bactericidal activity against Burkholderia pseudomallei (Bp), such as a Bp phage vB_BpP_HN01. A composition for preventing or treating an infectious disease caused by the Bp includes the phage as an active ingredient. The present disclosure further provides an antibiotic including the phage as an active ingredient, a feed additive composition including the phage as an active ingredient, and a disinfectant or a cleaning agent including the phage as an active ingredient. The present disclosure further provides a method for using the Bp. The purified phage has a specific bactericidal activity against the Bp. Therefore, the phage may be used for the prevention or treatment of an infectious disease caused by the Bp, and may also be widely used for the preparation of an antibiotic, a drug, a bactericide, an antiseptic, a disinfectant, and a diagnostic preparation for a bacterial infection.

### TECHNICAL FIELD

The present disclosure relates to the technical field of novel phages, in particular to a Burkholderia pseudomallei (Bp) phage vB_BpP_HN01 and use thereof.

### BACKGROUND

Burkholderia pseudomallei (Bp) is a Gram-negative saprophyte and a facultative intracellular pathogen. At present, a main problem in the treatment of Bp-induced diseases (melioidosis, also known as Whitmore's disease) is that Bp shows complicated intrinsic antibiotic resistance. Preliminary clinical data have shown that the Bp is resistant to penicillins, first/second-generation cephalosporins, macrolides, polymyxins, and aminoglycoside antibiotics. In a specific drug resistance mechanism, besides capable of secreting polysaccharide to form a biofilm to impede the effectiveness of antimicrobials, Bp may also encode β-lactamase, thereby eliminating the effects of β-lactam antibiotics such as cephalosporins and oxacillins. Moreover, the bacterium also has an efflux pump system composed of transport proteins, fusion proteins, and porins to further reduce the effect of antibiotics. Under the long-term action of a single antibiotic, the heterogeneous drug resistance of strains is difficult to avoid, and the accumulation of point mutations may also lead to the emergence of acquired drug resistance. Studies have found that a single base mutation in the endogenous β-lactamase may increase the resistance of a strain to ceftazidime by 85 times.

Currently, there are only three phages targeting Bp with clinical application potentials. Among them, phages ST79 (Umaporn Yordpratum, Unchalee Tattawasart, Surasakdi Wongratanacheewin, Rasana W. Sermswan, Novel lytic bacteriophages from soil that lyse Burkholderia pseudomallei, FEMS Microbiology Letters, Volume 314, Issue 1, January 2011, Pages 81-88) and E094 (Muangsombut V, Withatanung P, Chantratita N, Chareonsudjai S, Lim J, Galyov E E, Ottiwet O, Sengyee S, Janesomboon S, Loessner M J, Dunne M, Korbsrisate S. Rapid Clinical Screening of Burkholderia pseudomallei Colonies by A Bacteriophage Tail Fiber-Based Latex Agglutination Assay. Appl Environ Microbiol. 2021 May 26; 87(12):e0301920.doi:10.128/AEM.03019-20.Epub 2021 May 26.PMID:33811022; PMCID:PMC8174754) were isolated from Thailand, of which the former cracked 71% of tested Bp clinical strains, and the latter's genetic elements were used to develop serological diagnostic methods for the Bp. Phage C34 (Guang-Han O, Leang-Chung C, Vellasamy K M, Mariappan V, Li-Yen C, Vadivelu J. Experimental Phage Therapy for Burkholderia pseudomallei Infection. PLoS One. 2016 Jul. 7; 11(7):e0158213) was isolated from Malaysia and had a narrower lytic spectrum. However, animal experiments showed that under the intervention of phages, more than ¼ of the mice survived after being infected with the Bp.

### SUMMARY

In order to explore new methods for the diagnosis and treatment of melioidosis, the inventors of the present disclosure completed the sequencing of Bp HNBP001, a representative Hainan strain, in 2019 (Genbank number CP038805, CP038806). With this strain as a host, a series of phages that may infect Bp were isolated from 100 environmental samples (including paddy soil, irrigation water sources, and sewage) collected in Dongfang, Hainan in 2021. According to the shape of plaques, whether it may stably maintain a lytic cycle, and the ability to infect clinical isolates, the first phage vB_BpP_HN01 belonging to Podoviridae family with a broad-spectrum Bp lysis ability in China was isolated. The morphological characteristics, lysis characteristics, and infection characteristics of the phage were studied, and a whole genome of the phage was sequenced and subjected to necessary bioinformatics analysis. In addition, a cell model confirmed an in vitro anti-Bp ability of the phage and a possibility of treating melioidosis with antibiotics in the future, indicating that this phage has an extremely broad application prospect.

In a first aspect, the present disclosure provides a purified phage, having a genome comprising a double-stranded DNA with a nucleotide sequence set forth in SEQ ID NO: 1 and having an inhibitory activity against Burkholderia pseudomallei (Bp).

In a second aspect, the present disclosure provides a purified Bp phage vB_BpP_HN01, which was deposited on Apr. 15, 2022 in the China Center for Type Culture Collection (CCTCC) located in Wuhan University, Wuhan, China, with a deposit number of CCTCC NO: M2022422; the phage is used for inhibiting growth of a Bp strain; and the phage is used for control of melioidosis, a pulmonary infection, and/or bacteremia.

In a third aspect, the present disclosure provides a pharmaceutical composition for preventing and/or treating an infectious disease, where the infectious disease is caused by one or more Bp strains, and the phage according to first or second aspect is used as an active ingredient in the pharmaceutical composition.

In a fourth aspect, the present disclosure provides an antibiotic, including the phage according to the first or second aspect as an active ingredient.

In a fifth aspect, the present disclosure provides a disinfectant or a cleaning agent, including the phage according to the first or second aspect as an active ingredient.

In a sixth aspect, the present disclosure provides use of the phage according to the first or second aspect in preparation of a drug used in a method for diagnosing a causative agent of a bacterial infection, where the method includes:
(I) culturing a tissue sample from a patient;
(II) contacting a culture obtained in step (I) with a drug including the phage according to the first or second aspect; and
(III) monitoring an evidence for growth and/or lysis of the culture; wherein
the evidence of the lysis of the culture indicates that the culture comprises the Bp.

In a seventh aspect, the present disclosure provides use of the phage according to the first or second aspect in preparation of a drug, where the drug is used for reducing or inhibiting colonization or growth of the Bp on a biological surface in contact with the Bp, and the biological surface is selected from skin, damaged skin, and a mucous membrane of a mammal.

In an eighth aspect, the present disclosure provides use of an effective amount of the pharmaceutical composition according to the third aspect in preparation of a drug, where the drug is used for treating or reducing a bacterial infection in a subject in need, and the bacterial infection is an infection caused by the Bp.

In a ninth aspect, the present disclosure provides a non-therapeutic method for reducing or inhibiting colonization or growth of Bp on a solid surface, including: contacting the surface with the phage according to the first or second aspect.

### BRIEF DESCRIPTION OF THE DRAWINGS

FIG. 1 shows phage plaques after infecting Bp HNBP001 by the phage provided in an example of the present disclosure;

FIG. 2 shows an adsorption efficiency of the phage provided in an example of the present disclosure to Bp HNBP01 (FIG. 2A) and a final titer production of the phage at different multiplicity of infection (MOI) (FIG. 2B);

FIG. 3 shows a one-step growth curve of the phage provided in an example of the present disclosure (FIG. 3A) and lysis of the phage to host bacteria under the same conditions (FIG. 3B);

FIG. 4 shows a genome composition of the phage vB_BpP_HN01 provided in an example of the present disclosure, where repeat sequences and tRNA have been marked;

FIG. 5 shows an alignment result within a genome range of the phage vB_BpP_HN01 and a sequenced Bp phage provided in an example of the present disclosure; where FIG. 5A is homology comparison results with Podoviridae, Siphoviridae, and Myoviridae; FIG. 5B is a Viptree result; FIG. 5C is a homology comparison result based on a portal protein; and FIG. 5D is a homology comparison result based on an amino acid sequence of a terminase;

FIG. 6 shows results of a cell protection test of phages in an example of the present disclosure.

### DETAILED DESCRIPTION OF THE EMBODIMENTS

To make the objectives, technical solutions, and advantages of the present disclosure clearer, the present disclosure is further described in detail below with reference to the accompanying drawings and embodiments. It should be understood that the specific embodiments described herein are merely intended to explain the present application, rather than to limit the present application. In this application, the reagents not specified separately are conventional reagents, which may be obtained from commercial channels; the methods that are not specifically described in detail are routine experimental methods, which may be known from the prior art.

In a first aspect, examples of the present disclosure relate to a purified phage, where the phage has an antibacterial activity against one or more strains of a nosocomial pathogen Bp. The phage has a double-stranded DNA with a nucleotide sequence set forth in SEQ ID NO: 1.

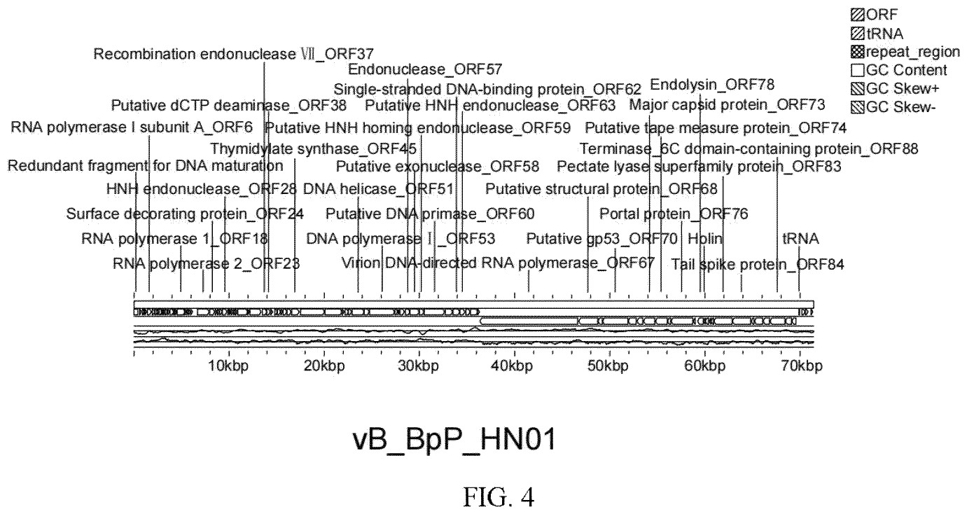

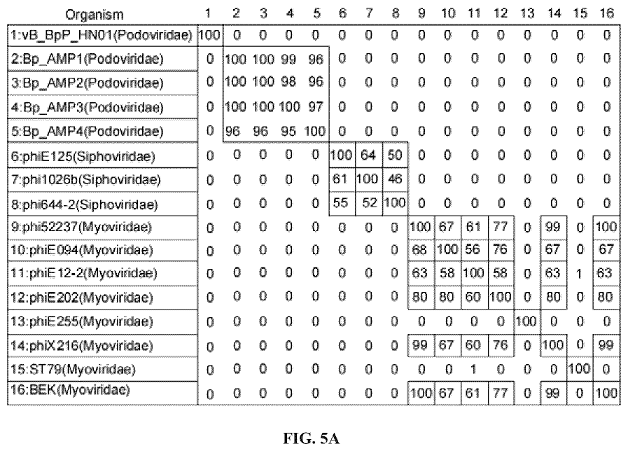

In a second aspect, the present disclosure provides a bacteriophage having a genome including a nucleotide sequence set forth in SEQ ID NO: 1 or consisting of a double-stranded DNA with the nucleotide sequence set forth in SEQ ID NO: 1. A specific example according to this example is an isolated Podoviridae phage (Burkholderia pseudomallei phage) vB_BpP_HN01, which targets the Bp. FIG. 4 shows a schematic structure of a vB_BpP_HN01 genome with a nucleotide sequence set forth in SEQ ID NO: 1. FIG. 4 further shows an open reading frame (ORF) in the vB_BpP_HN01 genome and its location within the genome, an amino acid sequence encoded by the ORF, homologous or similar proteins and conserved domains within an encoded polypeptide, and assignment of putative functional proteins.

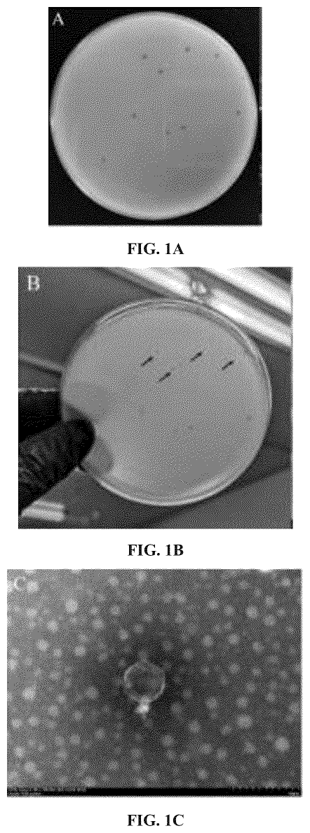

In the present disclosure, the phage belongs Podoviridae family, and has an icosahedral head with a diameter of about 62 nm and a tail with a length of about 20 nm (FIG. 1C). As shown in FIG. 1A, after infecting Bp HNBP001, the phage can form transparent plaques with a diameter of about 1 mm to 2 mm on a double-layer plate. If a culture time is further extended, the presence of haloes can be observed on a periphery of the plaques, suggesting the presence of elements for a capsular polysaccharide hydrolase on the genome of the phage (FIG. 1B).

The inventors of the present application have collected samples from an area with a high incidence of melioidosis in Dongfang, Hainan, and then obtained a total of 100 samples including paddy soil, orchard soil, chicken and duck manure, irrigation water, and sewage. Moreover, the phage of the present disclosure having a specific bactericidal activity to 25 strains of Bp and the above-mentioned characteristics was isolated, named as vB_BpP_HN01. The phage was deposited on Apr. 15, 2022 in the CCTCC located in Wuhan University, Wuhan, China, with a deposit number of CCTCCNO: M2022422. The phage is consistent with the information on a deposit certificate.

In another aspect, the present disclosure provides a pharmaceutical composition for preventing and/or treating an infectious disease, where the infectious disease is caused by one or more Bp strains, and the phage according to first or second aspect is used as an active ingredient in the pharmaceutical composition.

In some examples, specific strains of the Bp were shown in Table 1.

As used herein, the term “prevention” means all actions to curb or delay the progression of a disease by administering the pharmaceutical composition. As used herein, the term “treatment” means all actions in which the conditions of a patient have been improved or turned to favor by administering the pharmaceutical composition.

In the present disclosure, the pharmaceutical composition includes the vB_BpP_HN01 at not less than 10⁹ PFU/mL.

In the present disclosure, examples of preferred infectious diseases to which the pharmaceutical composition can be applied include but are not limited to: melioidosis with chills, high fever onset, pulmonary infections, bacteremia, and sepsis and other symptoms caused by the Bp.

In the present disclosure, the pharmaceutical composition may additionally include a pharmaceutically acceptable carrier and be formulated together with the carrier to provide antibiotics, drugs, bactericides, antiseptics, disinfectants, and diagnostic preparations for bacterial infections. The inventors can identify and extend as appropriate.

As used herein, the term “pharmaceutically acceptable carrier” means a carrier or diluent that does not cause significant irritation to the organism and does not abrogate the biological activity and properties of a compound being administered. For formulating the pharmaceutical composition into a liquid preparation, sterile and biocompatible pharmaceutically acceptable carriers can be used, such as saline, sterile water, Ringer's solution, buffered saline, albumin injection solution, dextrose solution, maltodextrin solution, glycerin, and ethanol. These substances may be used alone or in any combination thereof. Other conventional additives such as antioxidants, buffers, and bacteriostats can be added if desired. Further, diluents, dispersants, surfactants, binders, and lubricants may be additionally added to the pharmaceutical composition to prepare injectable formulations such as aqueous solutions, suspensions, and emulsions, or oral formulations such as pills, capsules, granules, and tablets.

In the present disclosure, the preventive or therapeutic pharmaceutical composition can be administered or sprayed to an affected area, or administered orally or parenterally. Parenteral administration may include intravenous, intraperitoneal, intramuscular, subcutaneous, or topical administration.

Dosages suitable for applying, nebulizing, or administering the pharmaceutical compositions of the present disclosure depend on a variety of factors. These factors include method of formulation, mode of administration, age, weight, gender, condition and diet of a patient or animal being treated, time of administration, route of administration, rate of excretion, and response sensitivity. A physician or veterinarian having ordinary skill in the art can readily determine and prescribe an effective amount of the pharmaceutical composition required.

In the present disclosure, examples of oral formulations suitable for the pharmaceutical composition include tablets, buccal tablets, lozenges, aqueous or emulsion suspensions, powders or granules, emulsions, hard or soft capsules, and syrups or elixirs. For formulations such as tablets and capsules, useful additives include binders such as lactose, sucrose, sorbitol, mannitol, starch, pullulan, cellulose, or gelatin; excipients such as dicalcium phosphate; disintegrants such as corn starch or sweet potato starch; and lubricants such as magnesium stearate, calcium stearate, sodium stearyl fumarate, or polyethylene glycol wax. For capsules, a liquid carrier such as lipid can be further used in addition to the above compounds.

In the present disclosure, for non-oral administration, the pharmaceutical composition can be formulated as subcutaneous, intravenous, or intramuscular injections and suppositories, or inhalable sprays through the respiratory tract, such as aerosols. The injection can be obtained by dissolving or suspending the pharmaceutical composition of the present disclosure together with a stabilizer or buffer in water, and then packing a resulting solution or suspension in an ampoule or vial unit. For sprays, such as aerosols, propellants for spraying water-dispersible concentrates or wet powders can be used in combination with additives.

On the other hand, examples of the present disclosure provide an antibiotic including the phage as an active ingredient.

As used herein, the term “antibiotic” means any drug administered to animals to kill pathogens, and the term is used herein as a general term for the antiseptics, bactericides, and antibacterial agents. The animals are mammals including human beings. Different from traditional antibiotics, the phage of the present disclosure has high specificity to the Bp, so as to kill specific pathogens without affecting beneficial bacteria. Meanwhile, the phage does not induce resistance, thus maintaining to be effective over sufficient spans of time.

In another aspect, the present disclosure provides a disinfectant or a cleaning agent using the phage of the first or second aspect as an active ingredient. To remove Bp, the disinfectant including phage as an active ingredient can also be sprayed and applied to any area including but not limited to livestock activities, slaughterhouses, livestock death sites, cooking spaces, and cooking facilities. Additionally, the cleaning agent including phage as an active ingredient can be used on the skin and body areas of live animals that have been or are potentially contaminated with Bp.

Hereinafter, the present disclosure will be described in more details with reference to Examples. However, these examples are for illustrative purposes only, and the present disclosure is not intended to be limited by these examples.

### Isolation and Purification of Phage

Dongfang, Hainan, an area with a high incidence of melioidosis in the province, was selected for sample collection, and a total of 100 samples were obtained from paddy soil, orchard soil, chicken and duck manure, irrigation water, and sewage. About 5 g of a solid sample was put into a sterile 50 mL centrifuge tube, 15 mL of 5M buffer was added, fully shaken to prepare a suspension, and then allowed to stand at 4° C. overnight to ensure the full release of phage. After centrifugation at 3,500 g for 20 min, a resulting supernatant was collected. 20 mL to 50 mL of a liquid sample was pipetted and mixed well with 20 μL of 5M CaCl₂, and centrifuged at 3,500 g for 20 min to collect the supernatant. The larger particles and some bacterial cells were removed by filtration with a 0.45 μm filter membrane, and an obtained filtrate was transferred to a new sterile 15 mL centrifuge tube and stored at 4° C. until use.

Before the isolation of phage, the Bp HNBP001 (Burkholderia pseudomallei HNBP001, BSL-2 Laboratory of Hainan Medical College (Key Laboratory of Tropical Translational Medicine of Ministry of Education and NHC Key Laboratory of Tropical Disease Control)) was revived: a single clone was selected and inoculated into 5 mL of a liquid LB medium, and incubated at 37° C. and 220 rpm overnight. On the next day, the bacterial cells were subcultured 1:10 into fresh LB medium and grown to the logarithmic phase. The phage was isolated according to a double-layer plate method: 10 mL of the phage-containing supernatant was added to 5 mL of 3×LB broth and 200 μL of Bp HNBP001, and incubated overnight at 37° C. and 220 rpm. A resulting culture was centrifuged at 4,000 rpm for 10 min. A resulting supernatant was collected and sterilized with a 0.22 μm filter. 200 μL of the filtered supernatant was mixed with 400 μL of Bp HNBP001 in logarithmic phase, and allowed to stand at room temperature for 10 min. 6 mL of a semi-solid agar medium was added (at 55° C., such that the medium was still molten) to a resulting mixture, and poured on a 2% agar plate. After an upper layer of the medium solidified, inverted culture was conducted at 37° C. overnight. One the next day, the presence of phage plaques, and the size and transparency of phage plaques were observed.

A single phage plaque was picked and placed into a 1.5 mL sterile Eppendorf tube containing 500 μL SM buffer. The phage was soaked overnight at 4° C. on a horizontal shaker, and then sterilized and filtered with a 0.22 μm filter membrane. 100 μL of the sterilized phage was mixed with 100 μL of Bp HNBP001 in logarithmic phase, allowed to stand at room temperature for 10 min, added with 6 mL of a semi-solid medium at 55° C. and mixed well, and poured into a 2% agar plate. After solidification, inverted culture was conducted overnight at 37° C. The formation of phage plaques was observed again. After repeating the above steps 5 times, the phages that could produce plaques of uniform size and transparency were stored. 5 mL of SM buffer was added to the phage plaque plate, allowed to stand overnight at 4° C., a resulting soaking solution was collected, and the plate was rinsed with 1 mL of SM buffer. The two were combined, collected in a 15 mL centrifuge tube, and the bacteria cells were removed with a 0.22 μm filter membrane again, and the remaining solution was used as a phage solution at 4° C. Phages that needed to be stored for a long time were placed in 30% glycerol and stored at −80° C.

In order to obtain enough phages for subsequent experiments, enough host bacteria were prepared: a single colony of Bp HNBP001 was picked from a fresh plate and cultured overnight in 5 mL of LB. On a second day, the bacterial cells were inoculated in 200 mL of liquid LB medium at a ratio of 1%. When an OD₆₀₀ (optical density at 600 nm) of the bacteria reached about 0.5, 4 mL of the phage solution obtained in the previous step was added, while CaCl₂ (5 mMol/L) and MgCl₂ (5 mMol/L) were added. Incubation was conducted at 37° C., 220 rpm until the medium was completely clear. In order to collect the phages therein, NaCl was first added with a final concentration of 0.5 Mol/L, and after being completely dissolved, they were allowed to stand in a refrigerator at 4° C. for 2 h. After centrifugation at 4,500 rpm for 20 min, a resulting supernatant was collected, PEG 8000 was added to achieve a final concentration of 10% (w/v), and after mixing, an obtained mixture was kept overnight at 4° C. The mixture was centrifuged at 4° C., 6,500 rpm for 20 min, a resulting supernatant was carefully discarded, and a resulting precipitate was resuspended with an appropriate amount of TM buffer, and filtered with a 0.22 μm filter membrane.

In order to obtain a specific titer, 100 μL of the above concentrated sample was diluted to 10⁻⁹ through a ten-fold dilution. 10 μL of the diluted sample was mixed with 100 μL of Bp HNBP001 in logarithmic phase. According to the double-layer plate method, when the generated phage plaques did not overlap with each other, the number (N) was counted, and the final number of phages was expressed as titer and calculated according to the following formula: titer (PFU/mL)=N×dilution factor×100.

### Determination of Host Spectrum of Phage

A single colony of the bacteria to be tested on the fresh plate was inoculated into 5 mL of LB medium, cultured overnight at 37° C., 220 rpm, and subcultured to the logarithmic phase at a ratio of 1%. On a 2nd day. 100 μL of the bacteria to be tested was added into 6 mL of semi-solid agar medium (55° C.), mixed well and poured into 2% solid medium. 2 μL of the phage with a titer of 1×10⁸ PFU/mL was added dropwise on the double-layer plate, dried, and incubated overnight at 37° C. upside down. On a 3rd day, according to the production of phage plaques, the sensitivity of the selected host bacteria was determined. The results were shown in Table 1. In Table 1, “+” indicated that the phage was capable of infecting a certain strain of bacteria and producing plaques, that is, the bacterial cells were lysed; the sensitivity to a bacterial strain that could not produce plaques was marked as “−”.

TABLE 1
Host spectrum range of this phage

| Clinical strain/ | | | |
|---|---|---|---|
| Strain line | Source | | Susceptibility |
| BpNo01 | Collect male patients who live in Dongfang | 57, Pulmonary infection and sepsis | + |
| BpNo02 | Collect male patients who live in Dongfang | 58, Pulmonary infection | + |
| BpNo03 | Collect male patients who live in Ledong | 31, Sepsis | + |
| BpNo04 | Collect male patients who live in Qiongzhong Collect | 39, Sepsis | + |
| BpNo05 | male patients who live in Ledong | 45, Sepsis | + |
| BpNo06 | Collect male patients who live in Danzhou | 60, Sepsis | + |
| BpNo07 | Collect male patients who live in Haikou | 51, Pulmonary infection and sepsis | + |
| BpNo08 | Collect male patients who live in Sanya | 67, Sepsis | + |
| BpNo09 | Collect male patients who live in Haikou | 57, Sepsis | + |
| BpNo10 | Collect male patients who live in Dingan | 66, sepsis | + |
| BpNo11 | Collect female patients who live inChangjiang | 30, Pulmonary infection and sepsis | + |
| BpNo12 | Collect male patients who live in Haikou | 80, Pulmonary infection and sepsis | + |
| BpNo13 | Collect male patients who live in Dongfang | 77, Pulmonary infection death | − |
| BpNo14 | Collect male patients who live in Dongfang | 52, Pulmonary infection | + |
| BpNo15 | Collect male patients who live in Wanning | 40, Sepsis | + |
| BpNo16 | Collect male patients who live in Danzhou | 54, Pulmonary infection and sepsis | + |
| BpNo17 | Collect male patients who live in Sanya | 49, Pulmonary infection and sepsis | + |
| BpNo18 | Collect male patients who live in Changjiang | 36, Pulmonary infection and sepsis | + |
| BpNo19 | Collect male patients who live in Haikou | 59, Sepsis | + |
| BpNo20 | Collect female patients who live in Wenchang | 55, Pulmonary infection and sepsis | + |
| BpNo21 | Collect male patients who live in Dongfang | 63, Pulmonary infection | + |
| BpNo22 | Collect male patients who live in Changjiang | 48, Sepsis | + |
| BpNo23 | Collect male patients who live in Wenchang | 48, Pulmonary infection and sepsis | + |
| BpNo24 | Collect male patients who live in Sanya | 64, Sepsis | + |
| BpNo25 | Collect male patients who live in Dongfang | 66, Pulmonary infection and sepsis | + |
| BcN1 | Burkholderia cepacia clinical strain ^{a} | | − |
| BcN2 | Burkholderia cepacia clinical strain ^{a} | | − |
| ATCC29212 | Enterococcus faecalis reference strain ^{a} | | − |
| ATCC25923 | Staphylococcus aureus reference strain ^{a} | | − |
| ATCC27853 | Pseudomonas aeruginosa reference strain ^{a} | | − |
| ATCC25922 | Escherichia coli reference strain ^{a} | | − |

^{a} The 2 strains of Burkholderia cepacia used this time and the common reference strains of Gram-positive/negative clinical bacteria were donated by the Department of Clinical Laboratory of the Second Affiliated Hospital of Hainan Medical College.

As shown in Table 1, besides Bp HNBP001, the host used in the attempts to isolate phage, the phage can also lyse 24 of selected Bp clinical strains. Only one strain (Bpn012) from Haikou appeared resistant. Moreover, for selected Burkholderia cepacia isolates and common reference strains of Gram-positive bacteria/negative clinical bacteria, the phage cannot initiate the lytic cycle or might transform into a lysogenic state after infection.

### Morphological Characteristics of Phage Observed by Electron Microscope

On the premise that the phage titer reached 10¹¹ PFU/mL, an appropriate amount of DNase I and RNase A was added to the concentrated sample, and incubated at 37° C. for 30 min. Residual endotoxin and other non-phage substances were removed with a 100 KDa ultrafiltration membrane (Amicon). 10 μL of the filtrate was added dropwise to a formvar/carbon supported copper grid (400 mesh), and absorbed for 10 min. The remaining sample was aspirated with filter paper, and negatively stained with 10 μL of phosphotungstic acid (2%, w/v) for 5 min. Excess dye was removed with filter paper. The prepared samples were completely dried at room temperature for 5 h, and then observed by transmission electron microscopy (TEM) at an accelerating voltage of 120 kV according to standard procedures.

The results were shown in FIGS. 1A-1C, after infecting Bp HNBP001, the phage can form transparent plaques with a diameter of about 1 mm to 2 mm on a double-layer plate (FIG. 1A). If a culture time is further extended, the presence of halo surrounding the plaques can be observed, suggesting that there were genes encoding capuslar hydrolases on its genome (FIG. 1B). A microscopic structure of the phage was observed by TEM, and it was confirmed that the phage was a typical short-tailed phage having an icosahedral head with a diameter of about 62 nm and a tail with a length of about 20 nm (FIG. 1C). For this reason, the selected and isolated phage was named vB_BpP_HN01, which targets the Bp.

### Adsorption Test of Phage

The Bp HNBP001 was cultured to the logarithmic phase, and the phage vB_BpP_HN01 with an MOI of 0.1 was added. 2×100 μL samples were selected at 0 min, 5 min, 10 min, 15 min, 20 min, 30 min, and 40 min separately. One part was centrifuged at 12,000 g at 4° C. for 5 min, a supernatant was collected, and a titer TA was determined according to the double-layer plate method. Another untreated sample was also assayed for titer TB according to the double-layer plate method. The adsorption rate was calculated according to the following formula, and an average of the adsorption rate was calculated by sampling three times at each time point. The whole experiment was repeated three times independently.

$$Adsorption rate ( \% ) = \frac{( {T}_{B} - {T}_{A} )}{{T}_{B}} \times 100 \%$$

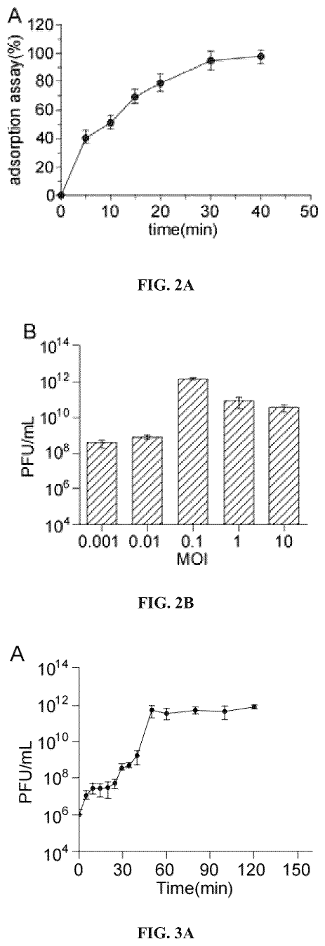

As shown in FIG. 2A, a phage infection experiment with MOI ranging from 0.001 to 10 was conducted to compare the final titers. It was found that when the MOI is 0.1, the phage can produce the highest titer of about 10¹² PFU/mL. And the adsorption rate was determined under this condition, as shown in FIG. 2B, about 40% of the phages were adsorbed after incubation for 5 min and more than 95% of the phages were adsorbed at 30 min.

### One-Step Growth Curve of Phage and Determination of Infection Rate to Host Bacteria

(1) The Bp HNBP001 was cultured to the logarithmic phase, and the phage vB_BpP_HN01 with an MOI of 0.1 was added to 10 mL of the bacterial solution, and incubated at 37° C. for 10 min to ensure the adsorption of the phage to the host. Centrifugation was conducted at 12,000 g for 10 min, the free phage in the supernatant was discarded, the bacterial cells were resuspended with 10 mL of LB, and incubated at 37° C. and 220 rpm. 100 μL of a resuspension was collected at 0 min, 5 min to 40 min (sampling interval 5 min), 50 min, 60 min, 80 min, 100 min, and 120 min separately. The titer was calculated, 3 samples were selected at each time point, and a mean was calculated. The whole experiment was repeated three times, and origin (version 7) was used to establish one-step growth curves.

(2) The Bp HNBP001 was cultured to OD₆₀₀=0.3, phages with MOI of 0 and 0.1 were added, and incubated at 37° C. and 220 rpm. The OD₆₀₀ was measured every 30 min, and an LB medium was used as a blank control. Three samples were taken at each time point, and an average of OD₆₀₀ was calculated. This experiment was repeated three times independently.

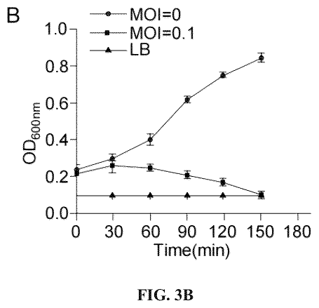

As shown in FIG. 3A, the infection of vB_BpP_HN01 to Bp HNBP001 was latent for the first 20 min, and then an increasing of titer was observed and lasted until 40 min. As shown in FIG. 3B, without the action of phage (MOI=0), the maximum OD₆₀₀ of the medium of the Bp HNBP001 strain reached 0.8 at 150 min. However, when vB_BpP_HN01 was inoculated at MOI=0.1, the growth curve of Bp HNBP001 cells decreased significantly at 30 min. Up to 150 min, more than 95% of the cells were lysed.

Whole Genome Sequencing and Bioinformatics Analysis of Phage vB_BpP_HN01

### (1) Genomic Nucleic Acid Extraction

10 mL of phage solution with a titer of 10¹² PFU/mL or above was prepared by expanding culture, and a genome was extracted according to a kit (ZP317, Zhuangmeng): 20 μL of RNase A (20 mg/mL) and 6 μL of DNase I were added into the solution to digest the host nucleic acid (incubated at 37° C. for 30 min). 5 mL of phage precipitation solution (pre-cooled in advance) was added, mixed well and placed on ice for 20 min. A resulting mixture was centrifuged at 12,000 rpm and 4° C. for 15 min, a precipitate was collected, and dried at room temperature for 2 min. The precipitate was gently pipetted with 400 μL of phage lysis buffer to resuspend, and 50 μL of SDS was added to disrupt the outer membrane of the phage. With an addition of 10 μL of proteinase K, incubation was performed at 56° C. for 60 min, then 500 μL of buffer B and 500 μL of absolute ethanol were added, and mixed thoroughly to precipitate the nucleic acid of the phage. The nucleic acid of the phage was adsorbed on a silica gel surface after centrifugation, and an obtained genome sample was desalted using 70% ethanol. After being eluted with ddH₂O, the concentration of genomic DNA thereof was measured, and the sample was finally stored at −20° C.

### (2) Whole-Genome Sequencing

The genomic nucleic acid of the phage was sent to Huitong Technology Biological Co., Ltd. (Guangdong, China), and the whole genome was sequenced on an illumina platform: a DNA library with an average length of 350 p was constructed with a NexteraXT DNA library preparation kit (Illumina, SanDiego, CA). DNA was sequenced on the Illumina Novaseq 6000 platform to obtain 150 bp paired-end sequences. The original sequence Reads were edited using an NGS QC Tool Kit (Version 2.3.3), and the high-quality reads were assembled using SPAdes software (Version 3.15.2). The possible ORFs contained in the genome were predicted using GeneMarks, and annotated according to gene homology through Blastx alignment (www.ncbi.nlm.nih.gov).

The sequencing results of the phage genome were analyzed using PhageTerm software. In addition, based on the similarity of terminase and portal protein, that crucial for the replication and assembly of the phage, a phylogenetic tree was constructed using MEGA (Version 7.0) by the Neighbor-Joining method.

As shown in FIG. 4, the vB_BpP_HN01 genome contained a double-stranded DNA with a nucleotide sequence set forth in SEQ ID NO: 1 after bioinformatics gene analysis. A repeat region with a size of 407 bp was identified as a starting point of the genome from the raw sequencing data. A total of 93 ORFs were returned in the preliminary search, and over one-third of them were not successfully annotated due to low sequence similarities. 22 ORFs were interpreted as hypothetical proteins due to the lack of homologs. Among all ORFs that could encode putative functional proteins, structural profiles of major components consistent with the microscopic morphology described above were established for 7 ORFs: an N4-like capsid was built by a unit translated by orf73; orf70 and orf90 contributed to the construction of tail, and its length was determined by orf74 and orf80, which were homologous to the tape measure proteins of one podovirus. On the surface, there should be a decorating protein (ORF24). 9 ORFs were found that are likely to participate in the nucleic acid-related events: the presence of ORF51, a putative DNA helicase, was a sign of the initiation of replication; the DNA duplex unwinding was likely to be stabilized by ORF62, a single strand DNA binding protein. When the primers generated by ORF60 attach to the DNA, synthesis would be accomplished by the polymerase (ORF53), with coordination by a dCTP deaminase (ORF38) and a thymidylate synthase (ORF45). In vB_BpP_HN01, ORF76, a MazG-like protein, could establish an environment ideal for phage reproduction through mimicking a nutrient-replete state. The transcription of macromolecules was solely dependent on its own RNA polymerase (ORF23 and ORF67).

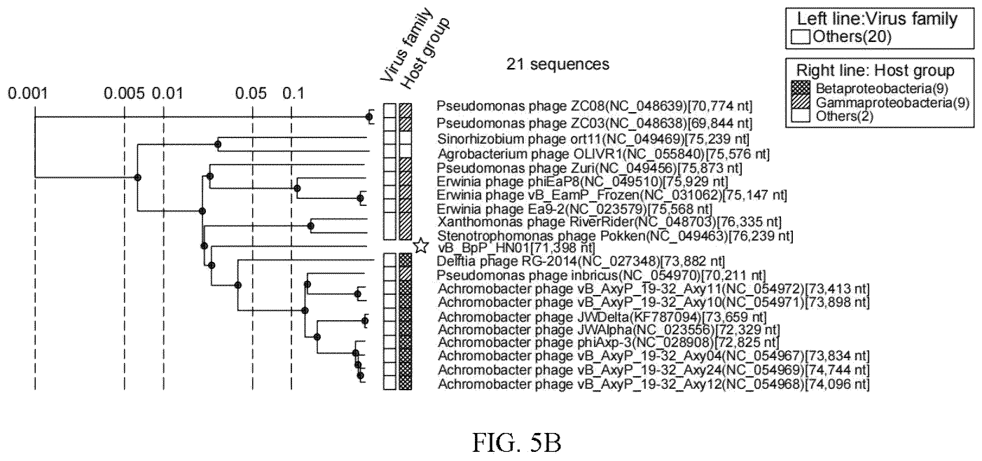

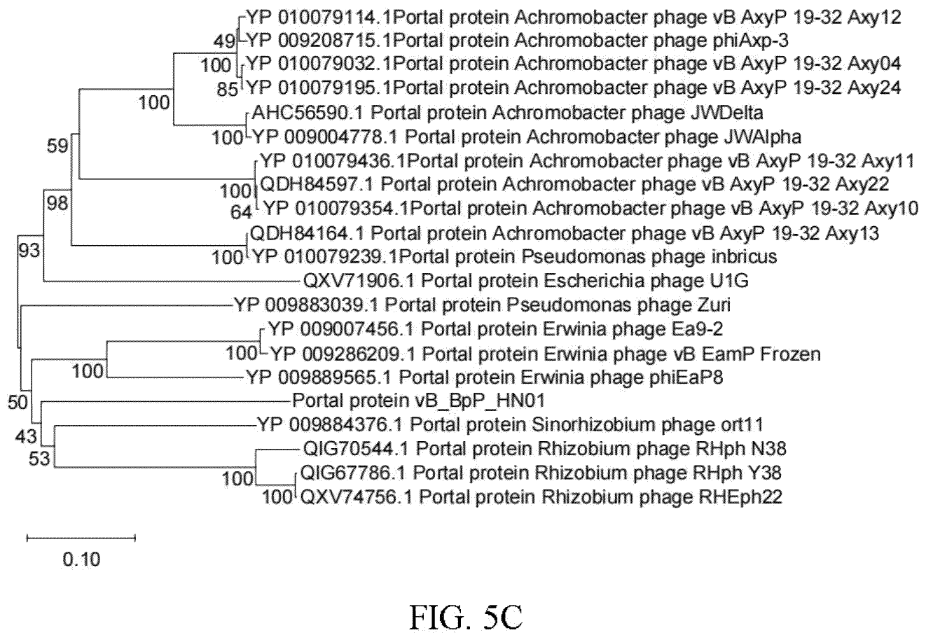

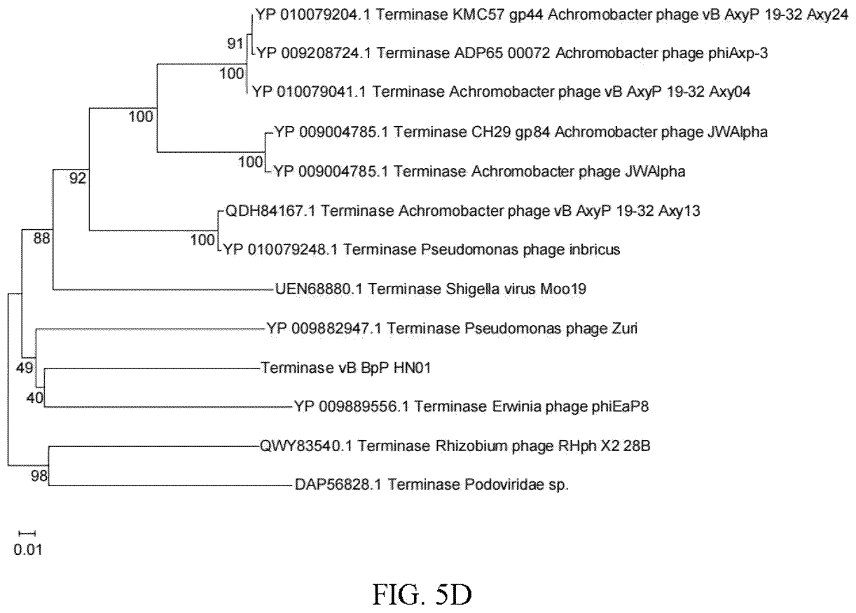

Further comparison of the whole genome of the phage found that, compared with the sequenced Bp phages (the relevant sequencing data was derived from www.millardlab.org), no homology of vB_BpP_HN01 was found (FIG. 5A). All the similar viruses/phages given by Blastn showed a total gene coverage of not more than 5% during the alignment, which were invalid results. As the virome is analyzed using VipTree, vB_BpP_HN01 was identified as a phage capable of infecting Achromobacter (FIG. 5B). Further analysis implied that this may due to the similarity of single genes or partial fragments such as RNA polymerase and tail fiber (FIG. 5C and FIG. 5D). Based on sequence similarities of the terminase and the portal protein, it was found that the phage might be a virus closely related to the Erwinia phage or the Rhizobium phage.

Cell Protection Experiment of Bacteriophage vB_BpP_HN01

A549 cells (ATCC® CCL-185™) were inoculated in RPMI 1640 complete medium containing 10% calf serum, and cultured at 37° C. and 5% CO₂. When the coverage of cells exceeded 70%, the cells were subcultured according to standard procedures. 2×10⁴ A549 cells were inoculated in a 96-well plate and infected with 2×10⁵ CFU Bp HNBP001. After 2 h, 2×10⁴ PFU of phage, 40 μg/mL of ceftazidime, and a mixture of the two (three groups in total) were added to corresponding wells, and incubated at 37° C. and 5% CO₂ for 18 h. Using CCK-8 kit, the optical density at 450 nm was determined according to the instructions, and recorded as A_{X}. A549 cells without any treatment were used as a negative control group, and their absorbance at 450 nm was also measured using the CCK-8 kit, denoted as A_{B}. A cell viability under Bp HNBP001 infection and after phage/antibiotic treatment was calculated according to the following formula.

Cell viability=(A_{B}−A_{X})/A_{B}×100%.

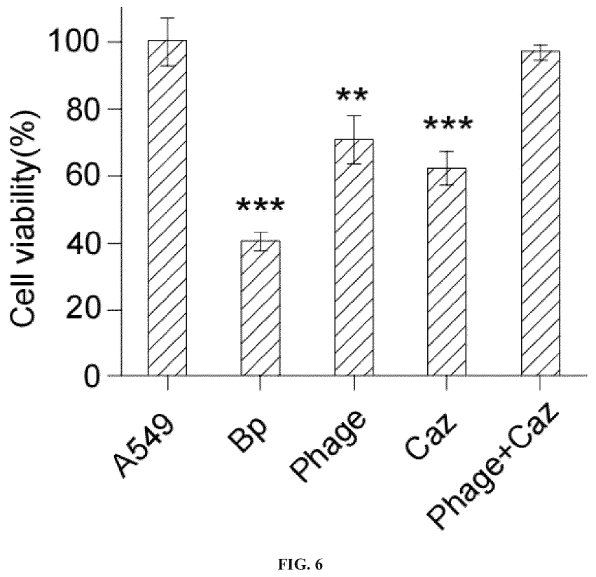

As shown in FIG. 6, under the action of Bp HNBP001, the cell viability of A549 cells decreases significantly, and was only about 40% of that of the non-infected group. However, after treatment with phage vB_BpP_HN01 at MOI=0.1, a cell death rate decreased, and a protective effect was slightly higher than that of 40 μg/mL ceftazidime. Moreover, if vB_BpP_HN01 and ceftazidime were used simultaneously after infection, the cell viability of A549 cells can be further improved.

What is claimed is:

1. A purified phage, having a genome comprising a double-stranded DNA with a nucleotide sequence set forth in SEQ ID NO: 1 and having an antagonistic activity against Burkholderia pseudomallei (Bp).

2. A purified Burkholderia pseudomallei (Bp) phage vB_BpP_HN01, wherein the phage is deposited on Apr. 15, 2022 in the China Center for Type Culture Collection (CCTCC) located in Wuhan University, Wuhan, China, with a deposit number of CCTCC NO: M2022422; the phage is used for antagonizing growth of a Bp strain, and the Bp strain comprises different Bp hosts; and the phage is used for preventing and treating melioidosis, a pulmonary infection, and/or bacteremia.

3. A pharmaceutical composition for preventing and/or treating an infectious disease, wherein the infectious disease is caused by one or more Bp strains, and the phage according to claim 1 or 2 is used as an active ingredient in the pharmaceutical composition.

4. An antibiotic, comprising the phage according to claim 1 or 2 as an active ingredient.

5. A disinfectant or a cleaning agent, comprising the phage according to claim 1 or 2 as an active ingredient.

6. Use of the phage according to claim 1 or 2 in preparation of a drug used in a method for diagnosing a causative agent of a bacterial infection, wherein the method comprises:
(I) culturing a tissue sample from a patient;
(II) contacting a culture obtained in step (I) with a drug comprising the phage according to any one of claims 1 to 3; and
(III) monitoring an evidence for growth and/or lysis of the culture; wherein
the evidence of the lysis of the culture indicates that the culture comprises the Bp.

7. Use of the phage according to claim 1 or 2 in preparation of a drug, wherein the drug is used for reducing or inhibiting colonization or growth of Bp on a biological surface in contact with the Bp, and the biological surface is skin, damaged skin, or a mucous membrane of a mammal.

8. Use of an effective amount of the pharmaceutical composition according to claim 3 in preparation of a drug, wherein the drug is used for treating or reducing a bacterial infection in a subject in need by administering the drug, and the bacterial infection is an infection caused by the Bp.

9. A non-therapeutic method for reducing or inhibiting colonization or growth of Bp on a solid surface, comprising contacting the surface with the phage according to claim 1 or 2.


SEQUENCE LISTING (1 sequence)

SEQ ID NO: 1 - DNA, 71398 bp, synthetic construct
  source 1..71398: mol_type = other DNA
gggggcgtccagccccctacacttacactacccccccggtatgtcttacacgtacacatggggggtgcccctatgttgtagatgctccgcatcttatggcaatcatgccttaactacataggagtctcatcatgtccgctaagctcactatcggtgctgtcctcggtacggttgcagatgcagcaggtgctatcagcaccactctcggtactgctaccaaggtagtcaacatcgctgacaactatgtcgaagactttgctaacaagcagaagattcgtattgctgcaagcaatgtcggatacaagaagcagatcatcatggaaactgctatgcaagttcagcaacagaagcttgttgttgataagttcgttgatgaaaatgactgtgcagatcatttcaacgatctcgtagcagaactcgaagcagcaatggcctaaacatggcctagacctcaaggagaaatccttggggtctaaaaccctttgagtggatagtccgaaggacgaactctcgaagagagttcagtcaacttgtagacggaaagttaaaccagagtctacttcctgtaggacaaatcctatggaaagtagaatcaaattagaggaaagacaatgcctttcatcaccaagatcatcctgatcctgatcgctcttgcgatcctcatcctgttcccgattgtgattccgttcgcaatctacatgctgctgagcatgggattcgaaggtaagaacttcggcaagttcaacatcactgatgcagcgaggcactgagtatggaatacgattgcaagcactggcctatattctgggaagtaggttatgagtttggtttggatacggatctatttatggatccacgagcagaagcacagtatcaacgttggctttatacgccgagtttgtcctctcaagtaaatgcatttgtacctctcggagactaaccatgagcatcgaagaacaactgaagctggctgaagaagaaatcgcagcatacgaagaggatcttcagtatctggatggtgaggacatcagcgatgaataacttttacgaactttactatcagtatgttcggagccaagaagagaaagaactcttcgcattctcatttaacgtgtggcgagactatttcggccacaacttcgagtaaatcatgaagaagataatcgctctcagccttgtcctgcttggcttagcaggatttggcttaattggtttgggtgcatacttcgtaactactccgcagcaatgggtagtgcccaatcaagacatggtactcatcacactgggtgcatgtgcagttggcttggtgatcctcatcgtgagccaagacaagtaatacagtcagtccaagtagtccacatttaatcgtttctcgggtatttagcccattcattattaatcctttaggagagtcatcatggcaatcaccaacgtttccgcattcggttcgtcgaagaggaccgcagcagcaaataagcaggaactcaagaagtcccaattctggatcaatatcggctatcaagctgagatccaaacggaagaaggaacggaactccgtttcatcagcctgccgaatggcatggcactagacgacatggaaccggtcaaggtgtccggtaacaacgaagtctggctgtatcagcagtccgcacgcaacgatctgctggcccaactgaaggccaaggcagagtcgctcaagccgggtgaggaagtcctcatcggtgaaggcccgatcgtgattcaactccgtcgtgtgagcgacgaagccaaggagatcaaggcagcagccaacccgttggcacgcaatctcggcctgtaagtaggcaagggggactgaccttcgggtcggttcccttaattttttagttggcttgagagttcaactttctaaagactaagttcaacttttaagccaattttgactaaaggagcctaagagctatggaatgtcatcaatgtggtgcttgctgtactgaaatcagtatttccacctcgtatgttggccatccgaacggtaagaaagcaggcactcgttgcatctggctggaggctgataacaagtgtttgctgttcaacaagccgctgagacctgccatctgtagcagctttcaagctgatcctgaaatctgtggaaccaatggggaagaagctaccaagctcatccgttggtatgaaaaggagactcgtccatgaagaagatcgccatctttgccctgttgatggtcagctttgcagtgcatgcagagcctcagatggaataccgcaagggtggtatcactatcgaactgaacaacagcaactggacgatgacgggtgataagtcgttcgacaaaggttgcttcgatcagaagatgggacgtaccaccatcgagcaacgcaagacgtacatctgttggttttacgacgtggacaagggtgacatcgtcctcaagttcccgcacacaaagcattttaagaggatctccatcaacgagttcgatgcagtctgagttagggaaatcgtatgaagatgcgtcaggtagacgacttcggtaatgagttctggcccatcttacacaaatgggcctactacactggtcgttggtattacgtgaccgtgaagaacaatctaatgtggtaactaacatttgagccaaggagagtgaacatgctaaaactgttcgctacgaacaaatatcatcgacgtggtggggccacttacgtgaatgtgatgcgtggaactccattgggcaatccgttcgtcatgaagcatgaaggcatgcgtgacgaagtggtggctaagttcagggcgtatttggctgaagccatagccaacaacgacgaggccatctgcgacgaactgaacagaatcgtagggctgttgcaacaggggcacacagtgcatctggagtgctgctgcaagccgaaagcttgtcacgctgatgtgatcgtagaagttatcaacgacgcagctaagtaatcaatcgaaccaaggggcgaaattatgctggtgcgaatcaatgaaggtaacggcaagttccgtgtcgtatggatggcacagaaggatgtggcacgcattctggagaatcagaagtgagcgaactcacgctgttcgaacgggagtatccaatggtagcaacggtagatgatgcattggccgttgctatcgaatgggatgagcgagccgcagtatgtaaggagaaacatctgatggccgctctgggacgtgcagtggcatggcgagaattggcattgagttatgccagctatctggagtttaagcaatgagagcattccttcgtagccaagacaagatgtctggccattggcccaatcgcacctacttcaacgtaggactgggtagtccgatcgacgtggagcgtcatgactatcctgagtggcttcaggagcaatgtaggaacagtgttcctgctgtggtggagtatctggacagcattgcacaagcagtgctggctggtggtgttatggcagtcgtgcttgaatacgagaccatgcactcacgttccaatgccatctggcttgtggagttcatcaacgacatcatcagtgcagccgaggagcaagtatgagctatcgccttagagtgaagttagaggtaatcaacgaggatggtgatgtggttgaccatcgtggactcgaccgattccacaaacgctttcaagcaccgggtatccagacccaacaggatctgttccgtacggacgatctggatctggcacgtgaacatctcaacaagatgtacgccatcatcggcaacatcaaggagcttctgaaatgaaaggctggaagaacgatggtagctgtgtgttcgtcttcggatcgaacgagggtggtaagcatggtgcaggtgcagctcgtactgcatacaacaagtacggtgctcgctggaactttagctatggtcacatgggcaactcgtttgccattccgaccaaggacttcaacatcagcaagacgctgagccttcgtcaaatccataactacgtgacgggtttcttggcatatgccgagggtcatccagatctcacgttttacgtgacgcgtattggctgtggtctggccggtctggaagataaggatatcgctcagatgttccacttcgcacccgctaattgcctcttcgatgaggaatggattccgtatctcggaaccaatggtcgcgaatactgggggacgttcgaatgaatgtatccattcaagacatcaaagaagcaatggcatggtggaatgctctcagccctgctgagatgctgaaacaccgtaagaaaattgaacgcaacgatgcaatctccattgctcgttactggcttaaggaaatcaaatgacagcagtatttgtggaagaagaccaagtggttaatgagatctatgcccttcgtacgggcatcggtcgcaagatcatggaaggtgacgagcaagcttgccgtaaccgagccaaggaaatccaagccacggctggcatgaagccgatgctcaagctggtcaagatcgtgaccatggaacaagaactggaaactctctaaggagaatcacatgacgacccataaagacgcgactgtagagcagtgggaacatcaggaactcatcgagaagactttcaacaagaaccagacctactcacgcattcgtgctgagttcgaggacgctggtgaagagttgaagcaacatatgatcgacaacggtattgaaccggatttcggttacgacctgctcgttcaaatggctctccacaagcgtgctgatgtagctacgctggttggtactctgcgtcatcacttcgctgaggcacaggatgtgctcaacatggcgatcaagtgtgcagaggctgatctcgtagagtttgtggtggatttcaaccagttcgtcgtaacactggacatctctgaagatgtgcaggaagaactggatcgcttccagtatccgctgccttgcatcatccagcctcgtaccgtcaattccaatacggacaatggctatcacttcgtccgtaagggcagtctgatactcaagcagaaccatcacaacgatgatatctgtctggatcacatcaaccgtgtcaatgctgttcgattccgtctggatcacgacacggccaagatggtgaagaaccaatggagcaatctcgacaagccgaaagaaggtgaggagaaggaagacttcgagaaacgtgtacgtgcattcgagaagtatgaccgtaccgcaaaggatgtgatggacttcatccaagagcttggcgatgagttctatctcacgcacaagtatgacaaacgtggtcggatctactgccaaggctatcacgtgtcctatcaaggggcaccgtggaacaaggcagtgatcgagttcgcagaaggagaaataccgacatgatttcgacccgtgcacagaccctgcttaataagtattcggaggctgcgagcatcgtttacttcctagaggatcaaggaactaacgatgctgaagtaaccaaagcgaaaaacgacttgcgtcaagctgagcaagatctcaagaaatacattgctatattggaggccaaatgagcaaggcaaagccttacgatgagtatgcagtcgaagggctgtataccatcaagagccacataaaaggcaacacgacaaaggagtggcgtgtgtgggtcggaaatatcaagacctacgaacaggctctggcaacgctcagaaatggacgtaaccgagtcttcgaatcgagctttcaactccgtatcgtcgggtacatcaaagagatcatcgaagatgagtcctgacaccagtggtcagtgggtgctacaacgtcctgacaccaaggagtattggaatacgggacacaagtggtatctcaaccaagctgcaatcctcagtgggagtcaaatggaagacttgatcggctccttcccctttctggtaaacgcataccacatcacaggaacctgaatcatgcctatctggctcctagtagcaatctccgtatttctgagtgcgtggatcgtctgtatgtatttcagcctcaaagatgacacggctcgcgaacgtcgtgcagccatgcttgggcgtaagtacgtaattacgtattcaccgctcgggaagttcttcctcatcggctggcttccgctcaccatcttctggctgagctacctgattttcggctacggcaagtaaactaaggagcaactaatgcggaaactgtccgtagttggcaacgtatacgggcagttaaccgtagttgctgaagcagagccacgtatgtccggtaaccggaaaacaccatacgtgctctgtagctgtacgtgtgggacatccattgaagttcttacatggtcactgaccaagggtttgaccgagtcatgtggatgtctcagacgtgaagttacaggagatcgtgcgagaacacatggtgagtcgcacacacggctgtaccacatctggaagcacatggtacagcgaaccacgaatccgaacaatgagaagtgggaatactacggtgaacgtgggatatccatctgtaaggagtggcgagactcttatgaggcattcgccaaatggtcacgcagtaacggctacgcagatcacctcaccatcgaacgcgatgatgtgaatggcgattacacccccttaaattgcagttgggccactcgtaaagagcaggccaacaacagacgcccaaggagaaagaaatcatgatgcaattcaccggcacgcagtatctgaagatcgacgtagcgaataacatgggtcacgacaaatggacatggaatgagcgaatcgaatgggtcgatgcacatgaccacgagttggaacaactgctggatcaagcagacgtaccggctctgtactacgcatccgtaaaggctctgcgtgcagcacaacgtggtgaagtatcgaacttccccatctcactggatgctacggcatcgggcatgcagattctgtctgcactcaccggtgaccgtaacggtgcagcactctgcaatgtggtcaaccgcattgtggatggtgaggtcaagcgtgctgacggctataccatcgtcttcgaagagatgaaggaacgtgtcggttcgtctgccgagatcaagcgtgacatgatcaagaaggcagtcatgaccgcactgtacggttcgaaggcaatgccgaagaaggtgttcggtgagggccgcctactggacatcttctacgagacgatgtccgatctggcaccggccgcatgggagttgaacgagcacttcttggagatctggaatccggagaacctgaagaactcgtgggttctacccgacaacttccatgtgcacgtcaaggtcatgacgaaggtcgaagaacaggttcatctgttcgacgagccgatcagcacgttccgtcaagtgaaccaaccgcaggaagaaggtcgctcactgggtgcgaataccatccactcgatcgatggcatgatcgttcgtgagatcactcgcatggctgactacgatgtgaaccaagtgaagaagttgtacagtctcaacactctggcactcgtggatccgatgagtctcaagccgttaaaggagtcgaaggagacgaagcttctacgtatcctgctgggtcacttccgtgactccggtttcctgtcggctcgtgtgatcgacatcatcaacgaagacaacttcgtgttgctggatgagcacgagcgtgttgccgtcatgaagttgattgatcaactgccaacgaagccgttcaaggtgatcgctgttcacgactgcttccgctgcctgccgaaccatggcaatgaactccgtcgactgtacaacctgcaactgcacaagctgtacgcgtcggacatgctgtcgttcctgttgagccagatcatgcaaacgaaggtttcgatcccgaaactggacaatgacatggctcaggacatcctcgacgctgagtacgcgttgtcgtagaatacggccaacttcctgtcaaggggagttggccaactttttaaatgataagggatagttatgacgacacttcaagtgtgctacaaaccggatggtcagaagatcatggtacaagagaatggcacggcagtacccggtggttacaccaagctgggtacgttcgatcacccggatcctgacgacagtctcagccgtaatggtgatagccatgtgtggtatcaccacattcaggagatcatgtaccacaccaagaactcgggtgctccgattgcagggttcttcccggagaacatcaccaacatgcagatgctgaccatcgaaacggacatcgttcgtgcagaaggtctgacagtggcaccgaagacagcaaatgtagctattgccgccacccagcaactgacgccgacgttcttccctgccggtacgacggataagaccgtgacgtacacaacttccgacgccacgaaggcaacggtgaacgctgctggtctggtaacgggtgttgctgttggtacggcaaccatcaccgttacgtccaacgatggtgacttcaaggacactactgtgattaccgttacggcataagtagttcttctctggtttttaagtcaacgatagcccctctccggaggggcttctcttttataggagacaaagatgacaacccgcgtcattcatattgctggaatggaactttcggggatccactatacagtggcatgcgagttcaatgcggcagaagggccaagcatttcgattcaagtagacaatcaagaaacacacggcatgtcaataactgaggctaatttactggctacagccatcaaaactgctattagtgtaattaaggaagaatgatgaacatgtacgtacacggcgagaacggatccacattcgtaggcccaatgatctacagcctctgcgactggcgtgctctggtccgatcgaaggcaatcgttcccaatgacggcatgggcttctggatcatcggtggcaaggtcagcgagatttcgtgcttcgatcccatgccggaaaacgcaacgggtgtcgaatggcacaacagatgaaccacaaagggggtgacaaagtgcacaagccatctgtcagcaaggtgcgtatcacactcaccccgtactggcagcagaagttcctgctcggtgcactacacgagcttggtgtgcatgagtcatacagtgtggacatgatcctgaccatgggtgaaaaggatttcgtccgcttctatacctctgccgcaaaagtagctgatgtcacaccgactcaattgaaattcgagtacgtgaacatcaccaatcaacccgtacgcattcgcatacgatctggtggactcaacaactatgatgaagacgcggcatagataaaggagaagcacatgaaagaacttacattggagcgtctgaaagaactgattagatacgatccgatcactggtgagttctttcgtattgcactgtctccgcgaagtaaggccaagctgggcttagtgaccttgtatgccaaggactctggacatcttcggatgtctattgatggtggtgagtattacgcagccagagttgcacactactacatgacaggtgattggggagagattgtggatcatcgcgacggcgaccccaacaataatcgctggttaaacctccgtaacacaacaccactaggcaatgtacagaatcaagtacgtgcacaccaccataacaagtcaggcttgcttggcgcacattggcgtgaagataaacaaaagtacgaatcggctgtttggcacaaatccaaacgttaccgcctcggttatttcgataccgcagaagcagcgcatgaggcatacaagcaaaagaagcgagagctccatgttacctgcacaatttgagccaaggaggctgagtaatggcgcaaggtgagacttttctcccgtttgtgattatgtaatcacgacgcaatcaaacacccatgagcctgactcattggctcatctggcaatgtgctgcacgaacacatagcgatgcaatcgaagccttcgaagaagaaaatccggactgcgagttcattgaagtagtgcagacggctgacttcaataaagccattcaagaagcggaggctacataccgtggctaaggaatacgtcaaggactctgagggtgaaatgatcgagaagatcaagcctgagaccaagaagtacgaagagtcttaccgcaagttcatggagtactacgtacctgcacacaaggattgcaatcattgtggtgctgtgcgtcacaaaaactaccggtgtgtccggtgtggatacgaatgatgaccactctccagcttggtcagcgagggatcctcgctaacaagaatggcacgctgatgtgtgccaccgttctgtctcttggctcgaactacgatgcatatcgtatcgacgaccagaagactcctaaacgtgttccgaaaggaaccaagacctacaagttcttcgttggtgagtccgcagtggtcgatgcatcgatttggattcacacgaggggcatcaaggaactcgatcatgcctgaagcacgaaagacatctgttgaactcctgctggaactcaatcagatgacacgcaagtacgacgacatcaccaagaccggccgtatcaagatcggcactgtggcgatggcacaagagatcgtcatcaagtcggcagagttgttggctgctacccgtcacgagtatcacgaacaaaacccggtggactgatgagcaagcgaaacagtggtcaaaccacacgtctggtgctgttctacgtacagcagtgtttggaacatccgagcgatgaaatcgccgtgtccgatcaccctgccgagaacgcagtccatgcgaccatccatcagaatctgatggtggcgaacaaggtcagtgcaatccttaccgcactgaatgtccaacactacgtgaacagcaacaaggtttgcgtgctgccgatcaacaaagaccaatgagcgaagtcgctgaaagccccctctcgctgtaccagaacggtacggctgatcaagtctacaacgacctgatccgtgtgctgcgtgccaaccgtgttgccatgatccatggctcaccgggtattggtaagtcagccattggccataaagctgctgaagagtgcaacctgctgatgcttgaccatcgcatgagcacgtcccgtccggaagacctgactggtctgccgcagttcaaggacggctatgcacgctttgcacccttccgcgaactgttcccgctggaagatgcagaactgcccgagggcaaagatggctggctgctgttcttggacgagttcaactcggcccacaagagcgtacaggctgctgcatacaagctgatcctcgaccgtatgatcggtcagcacaaactgcatccgaacgttcgcatcgtctgtgctggcaacttggcaaccgaccgtgccatcgtgaacgatctgggtactgctatgcagtcccgtctggtgcatctggagatgcgtgtcgacttcgagtcgtggctgaaaaacgtggccattccgttcaagtacgacagccgcatcgttgcgtatctgagccgttacaacgcaaagctgtacgacttccgtccggatcacaacgacaagacgttctgctgtccgcgtacgtgggaattcgtcaacgatctcatcaaggacgaagcagcactggacatggctaaggatgctcgcctgctggctggcaccattacgtccggtgtggccaccgacttcatcgaattcacgaaagtgttcgagaaccttccttcgctggaagaggtcgaggctgatccgtgcaacgcgaagatggcaacggatacgggaaccaagtgggcaatcaccgcatggctctcggagaaggctgacgaaaagaacttcggtgcaatcacggagtacatggatcgcatggacaccacgttcaagatcctgttctaccgcatgatcgtgctgcgtcaaccgggcctaaagatgcatcccgcattcagcaaggctctttcgtccatcggcaagtacctctggggcaacgtctaatggccaagttcctgaacgagtgcaagcatggtgatcacacgtacatcagcgtcttcgatgcggatgctgtgatcatctatgctcgttcgatgccgcaagctcaccagattgccatcgagcatttcaaacccaagaagcgagacaagaagctcatcgagaccaagctgcatatcatgagcaaggacttgatgaagcacgtggaacgcttcgcaaactacgaaggaaacgcgtgaaaccgaccccggtagttgattccgagttcgaccaatacgaactcactcgtgagctggacaaaacgaaatcggaagtcttcatgtcggggtcttccgccttctacgggccaatcatgtgctcgctggagttcatgtggtcacgtgatatccagacggctgcaacggatggtgtgcacctgtactggtcggacaagtttttcatgaagaccaagccggtgactcgcaagtgcaccctgcgtcacgaactggagcatgtcggaagactgcaccgtttgcgtggcatcggtaaagatcccagagactggaacatcgcgtgcgattacatcatcaacaacgcgatgaagaaggaaggttgggagttcgatccggacgacatcaaggtgtacatcgacgacgactatgcggacttgtccgaggaagaagtctacgagttgattcactccggaaagatcgagcctaagcttccgccgggggccaatctcgatgacgacatgctggaggaagaagaaccaactccagaagagatagccgcaaacatctcaatcgttgtgcaggccatccatgctgcaaagatgtctggcaatccgggagcaattcccgggtgtgtcgagcaagtaatcgacagattcctgaatcctgtgattccatgggaaaaggaactcgccaagttcatgacggagcttcaggacttctactacacgtgggcacgtccggaacgtcgttacatgagccaagacatctatcttccgagtatggaggatgacgaaggagcattggagcatctgtgctactaccttgatgtgtctggttccatcacagacaaggacgtgattcgcttcaacagcgaggtcaagtacatcaaggaacagcttcgacccaagaagttgacactggtgctattcgacacaatcattcaacaggaaattgtcttcaatgaggatgacccctttgaccgtgtggttgtggtgggccggggtggtacatgtttggcacccgtacgcgagcacatcgagaagaacaaaccaaccgccgctgtcattttttccgaccttgaggtcacgcccatggttaaaccttcccgcaacatccctatactttgggtgcggatcggtaacactgggcacaagccttccttcgggaagatcataaccatccggagttaataattgtcggcacaactggagaaacagtgccggaagtgccttcaatcatttccggcaacaaccgaattcttttacaggaattccggtggcaaatacggactcacgccaagatgtaagtggtgtgtgaacatggataacatggaaagccacgttcgccgtttggccgctgatcctgtaaagatcaaggcgcaagctgctgcaagagccgcaaaaagttaccacaagcacttggagaagaatcgagccaagggaagggaacacgctgccgctgccagagccgatcctgagaaacgtgcaaagatctacgcacgtaagcgtgcaggcggggctgggcttacagtagaagagattgagcagatcaagcagaagcaaggcaacatgtgtgctatctgcacaagccctaatcccacagacttggatcactgccacaagaccggtgctgtacgattcctgctctgccagcactgcaaccgtggtttgggggcattcaaggacaaccctactctgctgcgaaaagcagcggatctcatggaagagattcatgatagtcaatcaaagctcactgttgaaagcagccccgatcaagggcatggttcaggagaaacgtaagcacttgggcttctcgtggggtctgacggaatgcggatacgacatccgtatcaagcaatctgtaactctttttctgggtcgtcgttttcggcttgcatcctctatcgaggaattcgatatgccacatcacttaatgggccgtgtgctgaacaaaagcacatgggcacggaaaggtgtggatgccagcatgacaactaacgtcgaaccggggtggaaaggatttttgaccatcgaactgcactacaagggatggaagccacttaccatccctgccggtgtgggtatcgcgcaagtgatcttcgagaccatcgaagttcctgtcgtctacgatggcaagtaccagaaccaagaggatcgacccgtagaggctcgtgggtagtagacacgcagcctttgtttggggtacagtaaacacgtaccaacaacggagaccatgtaatgctgcaagtgcttctgatcctgaagctcttgttcggctgtgaagcaaagaccctgagtatccagtctgtccgctggtttgcgtcaagagctactcaggataaggagggcagaagccctcacgtaaccgagattaatcaactagaatacggttagtaacgcttcacatacggacatagagaacataggatgatagggtgatgaccaaccatcaaacatgtggagtgctcttgcgaaagcgagaccccacccgagtccaaccgcaaatcgggggattcggaagaccgagagacactttgtcgttgctgtccccaacagccggtctttcaaatcccctctacccaacaggcccatgctagtacagatgggccttttctatttctgagccatgattactcaagacgaagtgaaacagatagcggaagccctgaccgttgctgagggtcaggttctgctggaggtacgtgatagccaagggtggactgtgaaatcccttggtgagattgcagaagacacctgcatgtccatcaataccgttcgacgtacgattaaatctctacgtgagaaaaatctcgtcgaactcatccgtctgacgcaagacgaatacgaccataccttgtgtggctctgggtatgcacgaacctacgcgggtgaccgtgttgctacggagatcagccagtcatgatcagcaacacttcagggatgttgttacttcgttggatgggtaaagaaggtggttatgagcttcatgaccatgcctaccatttcacatcagacgacgttgacatgtactacgacgccatttatgacgccgtagtggaacacctcaatagcgtaaagcctgtactggccatcgacgatgtgcttcgtatcgtgttcatcggagtgtactcactagttggagatggtgaggatgtggatgagttctttgatatcgaagcaattcactacagaaagatggacatggacgaactcaatcgcttctacgagcaactggaaatcgacgaggacttacgtgaacaagaaagagcagaagaagatgcgtcctgactatgtcatcctattcattgatgacttggaacctgctgacgtaaagcaagcaatcgacgaatgtcagcaaggagcactgctgggtgcattcgatatcgcggaaacacaggctgatgtgcataacgcaaatgaacatgcacgtctcactcaacgaaccaagaaacctttgccgtactatcacggcaataaacggagattttgatggaacacgaaattttcaccgcagtaatggttggtatggctctgtgcgtgtctggattctgtaagcactccacacgcgtgttcgtgggcttcctgatcattggtgtagcaggtatggcctacagcaccaagagccatgcagcagactggggtaatggtctcaattgcgattcgatcgctaacctcagccgtgctgtcatgtaccagaaggagcagggcaacgaccggtacacgctgaagcagatgacgtactacgcagcagttggtgcaggtgtgaccaacctcaagctgcttaccgatgccatcgtcgactcggcattcaagctggagggcatgactccgaatctgttctcggcaatcatgtacaacgaatgcgttcgtgccaatcaggacgtacgtaagcatggggaaggcatgctgtaatgcgtaagggaaccaagcgaaagatacgtgttgccagcaccccgatagtctttcgtgatcgtgctaatgaggcagaactgatgcttattccgcatctatgccttgagagcttcacgaagggactcggcaatgagcaagcattcgatacgcttacatatcgaatgctaatggcgacagcactgaccaagcacatcgaggttgaggaagacttcgattacgcagcaggggtaattggtgcagcagtacaagccatgaagaacatcggtgaccgataccttcgcacaggtaaatggggtatgagtggtggcgacgtgagccgtatcaaggacggtttaggtatcgctgaccaacttcaggaactggtgccgattaaacagcagtctctcgcttcgcagaaggttggcaagcatgtcattggctcgatacgtgagactgtccacaacctcgaaagacttctggagaagaaatgaatcagcacactggcgagcgagcatacctcgcagcattggagcacatcatgatccatggttcgcattacatggatcgcaccaaggtgggccgcaagtccatcaaagggatgatgatgcgtttcaacatggccgatggcttcccgatcatgaccacgaaaaaggtgagcttccatgctatcaagcacgagcttatctggttcctgaatggcgagaccaatatcggttacctgaagaagcatggtgtgaagatctgggatgaatgggctgatggtcatggtgcactcgggccgatttacggtgcacagtggcgtacgtggccgaagcaagtcgacgggcacacggagtacatcgaccaagtcggtgaactcatgcgttcactgcgacacaatccgttctcatcccgcaacctcgtgtcggcatggaacgtagctgaactcgaagacatgaagcttccgccgtgtcacttcgcatggcaggtgcatgctcgccaagaagtcgacggtatctatctggatctccagttgtaccaacgatcggctgacatgttcctcggtgttccgttcaacatcgccagctactcgttgctgctgcacatgatctgtcatcagctcaactaccttccgggtgagttcatctggactggtggtgactgtcacatctatgacaaccatcacgaggctgtggacgagcaactgcaacgtacgccgggagcattcccttcgctgctgatcaaccgtgctgcaccaaacatcttcagctatgacgctgacgatttcgaactgctggggtatctgccccagcctgccatcaaggcacctgtagccgtgtaaggctacgcaccctttcgagggtgcttatacgtggagacgggtgcgacccaaagtggaatccgctcgcaccggaggatatggagggccgtctcccagtataagcatcactcccaaccaactgaaccaaggaaaatcacatggaagtcaataggccggaaacactgatcacccacgctgttctcggtggtggtcaatcgcaggaatttggtatttcgaactcggcagagttcttccaaatcctttcgtccacgctgtacaccgatcagaaaggtgctgttgctcgcgaagtgctctgcaatgcatgggatgcacatatcgctgctggcaagactgacatgccgatcgagcttacgctcaatcgcgaagaattcgtcattcgtgatttcggctttggcatcgctgatgccgatatcgggccgatctacatggtgtatggcaactccaccaagaagaacgacgggaagcagaccggtggtttcggtctcggctgtaaggccccgttcgcctacaccgaccacttcaaggtgacgagcatcaacaacggtatcaagtccatctacaccatgtcgaagtcggctggtacggtgggcggcaagccgggttgcactcgaatcatgcaggctccgactgacgaaccgtctggtctggaagttcgcattcccatcaactcaacgggtgacgtggctgagttcaagcacaacatcttcaagttcgtgcgtaacggtgagatgctggctcgtctcaacggcaatccggatacgatggaaacgctcccgttctcgacgatgaagcatggcttcctcatcacggatgtggacatcggtttcaatgacccgatccaagtacgctacggcaacgtgatctatccgattccggatcatgagagctatgcccctcagtacaagcagattcgtgcactgctgaagaagttcgggaagcgtgattactacaatagcggcatcttccgaatcatcctgcaagcacccccgcactcgatcgcagtcaccccatctcgggaagctctgtcgatgcaggagcacaccgtgaactcgctgaagagtctgctgaacaacttcgtgtccaccatcaacgtggatatgaaggagtacgtgtacaagacgatgcgtgagtcgatcgccagtgcggtgtccaaggcttcctacaaggaactgctgtcggatgaccagaagatccccggcctgaatgcagacacgctggcacgcaacaagtatgtggtcgatacggaatcgatctccaagcacgtcgtgtatcacaactacccggactatgatggcttccgtaaggaagaggtcaaactccgtgtgatctcgatgattaaggctaaccaagtccaccgtggcttggctcaaacgtggctggatgcctacgatgccgagccggagaacgtacgttcgggtggttggtacaacaactcgcatagggctgatccaaagtcgtggttccagcgtaatgtcgttggcctgttccgtcgcaagatggtagcagagccggaactcgacatgcaacgacttctgacgtatggacatgggaaccacgaggctgcacgctatggcaatacgcatggtgtcaccatcgcccacatgcagtccaagactccgttggaaagcctgttgccgtatctacgcaacatggtaatcctgtcgttctcgcgactggaaattggccctcgtctcaagagctatgaattcccggcagggaaggattgggagccgtcgtgctgtcttatgtacgtggttccgcgtagctcgcagaagaagatcgaggctgctcgcaacttcttcaccaacatgggctgctacatcgttgacctgaccattcgtcattcatgggaagatcagtcagtgctggccgtggagaagaagactgttgttaagaagccagtgaagaagggtctgccgattctcgcagacggttacgattcaggtggctatctggcaatgcagcttctgcgtgacaaggaagacgctcgtcgtaccgaatcaccggaatggatcgtgcgactccctaccatgtccagtagtggccgtattagcaaggggtgctacgatcaatcgctcggcatgtttgacgaaacccaatcgaagctcatcgtgaagctgtggggcgacgttggtggtggttgcacttccgagaagcaaatggaatcctaccattccaaaggggccaaaacagtcgatgagttcgtgaaggaaaaggttcttcactccatgaagaccaacgttgcaatccggagatacatgaagtttgctctgtggcgtgtctttggttcagaggaatctcgtcgctatgacaataagcaacaagtcatggctgagtttgccaagatcgtctacaacacgcccgaactcgccaaggagtttggcattgtggacacccgaaccaaggaagagaagtggatggtctcgctgtaccgtcacctcgtctccaatctgtattggatcgaaaagaagttcccggagatcaagactgtgatggatagtatcaacaccatctctcttgatgatggggcgctaaagctgaagaaaaccctgtcggaaggctccactctggaattcctcagtgcagaacagatcagcgaagcaatcaagcagaaagtagacacggcacgtcgtgatcaggcaattgccttcatgaaactcgcactcaaactttaaggagcagtaccaacatgactcacaaaatcgtagttgtgtcggctgttgtcgacacgttccggatgacgctctacacccagacgggtgcaacgcatgtcatcaaccaaggcgacccgaatctggagaagattctagccatcatcaccccgaaactgtcggttaacgaaccggtggaactcgacctgacggctctcaatgccgagaagccgatcaacgagtattcggagatggagaagaagacgaatggtttcgtccgattcttcaaggtggccaaggagaagatcagttcgatctttggctcggaagagaagcagaagacacccgaggtaaagcatgtcgcaagccagactcttggcaaggccagcgaagacatcaagccgaccattgcggctacgccaacggttgtggatgctaagactgaggctgtcgcgaagaaccaagcggcagtcgctgaagttctgaagaacgcaaccccagtgaaggacgatccggagccggtcaaggcaacggagacgctggtcgctgtggtcaaggactcgaagggcaatgatcaggtgatcgccggtgtggaagcactgtcgaaccaactgaagcactcgatccgtggctcggagatcggtatccagaacttcctgaagcgtctggccgaagtcatcagccagcgtcgccactcggtcgacgacctgatgaagttcatggagcgtggtgatctgcctgtcagcgatgctggtgacatcatcatctacaagatcctgaacaaatgtaagacgaagctcgaaggtatcgagttcgactacgtcgatgtccattcgtcacgtgtcgtgcagcgtgtcggaacgtacatccacatggatcattcgctggtcgaccacgaccgtcgtaacgagtgttcgaacggcctccacgtggctcgtcgtcagtatctcggtggctttagtggcaacgtaatcgtgctggctcgtgttcgcccggaagatgtcatcgccgtaccacaatacgatgccaacaagatgcgtgtctgtggttaccacatcctgttcgagatgctggaagaagaccgtcgtgcactgatcagcaacaagccgctggtcaccgaagacggcaaggccaagctcggtaaggcactggccggtgatcacgtgggcattctcgaacacgtgaagatcggtggagccaagggcaccaatctcaccgtcaccaagaaggtgaagggtgagcgtgtggccgacgaactccagtcgaacgtggtgcaggcaggtgatcgtgtggaatcggctatgcaagtcgatgacgacgacaagctccaagctcctgctgtggatccgaagaccatcgcacaggtggcagacgcagccaaggtagccgtggcagcagaagccccgctgtcgcgtgcagaacaggctcagaagctttggaacacggtcaacagcactgccaccatgggcgttcgtgcaacggctctggagacgcttatcgagctgaaaaagaaggcgaaggtgtcgtggaactcgctgggtctgaaagaccacgagatcaacgagcagtcgagcaagctcctgaagcaaaacaaagtggccgaagcaaaggtcgctcccgcaaccgcagttaaagaagcgaaggaaggaaaagtggcatctacgcaagcatcgggtactccccgtgaacgtatcgcagcactgctggccatgccgggtgcgatcgatcgtctccgtgccaagcagatctatggcatcaagcaagacgccaagaagggctggtcggccctcggtgtgacggatagcagcatcatcaagaccatcgagaagaaggcgaaggactgatccatgggggcaaagaagaacatgcccccggacgatcatcgcctcacattcggcaagtacagggggcaaaccatcggatacgttcgatcggttaaccccctgtaccttgagtgggcgagcgagaacatcgccgggttcaaagacggtcagcatggcaccagaccatcggagcttgctcaccgtcgcatcattgccaaggtgatcacacactctggcaaggttgagtggtacacggagtgataatatagagtcttctttaaggaggctcaaatgtctactgtgtatcgccctaatcgaaaggcgacagaccatgacattattcgcatgaatagtgtcggtctgtcccttcaaaccatcgcccgtatcatcgggtgtcaccccaccaccgtgaccctgcgtctcaagagtctccagattccacctgcagacacacgacgcacgttcatggaggacattttcaagtcactcacccctgatcatcaggagtggctggccgatcggcttggcccgactgaatcgatcaaatggtacgtgcgtgacttgctcgtagaggaatacaagcagcatggtcacaatcaaacaagtaatcgactggttcgccaaggcacgtcccaaccgaaccaagcagcagttcctgtcgcagacgggagtgcacttcgaggagatctcggagatgctggagcagatggctcctgaagatagccatgcagcacggctgattcatgacgcctacgtggcgaacaaggcactcggccaagccctcaaggcaggtgagtgcagtttccgagtcaagaacgatgtcctgttcttggacgccctgattgaccagatcgttacggcgactggcaccggctacggcaagggatacaacatggatggtgcactcgaagaagtgaacgcatccaactggtcgaagttcgacggggagaccccgatcttccaagacaacgacccggacaagaagatcctgaaaggcccgaactacttcaaggccgatctcagcagtttcatctaagccccgaaaggggcttttcttattggagccaaggaaatatgacgaccctgaacgcagggcaacaagccgcagcagatgggatcttcgatttcctgctcggtaatgagaaggaactcgtgatcagtggccccgggggtacgggtaagacccacctcatgggccacatcatcgacgaagtcctgccgatgtaccacaagacgtgttcgatgatgaacatgcccccgttgtaccgtgatgtgtacatgacagccacgacgaacaaggcagcagacgttctcggcagcagcactggtcgacccacgtcgactatctacagcctgatgaacctgaaggtcgaacagaacctcaacaatggttcgcagcgtgtcaccaagacgaagtcttggatggtgcacaggaacaaaatcattttcgtcgacgaggcttcgtttgaggattcggtgctacgcaactacatacttgaaggtacgaacaactgcaagattatccatgttggagaccattgccagttggcaccgatcatggagcagaagccccctgtattctcagcaggactccccttctacgaactcaccgagcaaatgcgaaccagcgatgccaatctgcaaggattgaatcagcagcttcgccgtaccgttgaaacgggtgagtttctccccatccaacacgtgccgggagtcatcgactgggtgacggacgaggatgagatgcaggctctggtggaggaacactttgtggatagtcaccacaactcccgactgttggcatacacgaacgatcgagtgaacctgtacaacgggttcatccgtgacttccgtcagttgcccacatgtccggtggagggtgacatcctcgtgtgcaactcggcaatccagttgaataccgtgcagaagtcgatccccacggaaacgcagttggagatcgagaagatccattcgcagtacaaggtgacgatcagtccggatgtggagttggatgtgttgctggtgaacgctcgttcgcagtatgcactctacgagaatctggaaatcccagtcgaccgtgagcacttcaacgccctcaccaagtggtatgcaggtgagaagaactgggaacgctacttctatctgaagaacacgttccctgatctccgtccggccgatgcgtgcacaacgcacaaggctcagggcagtacgttcgataccgtgttcatagacatgggcgacatgagcaactgtcgcaatccggacacggcagcacgcttgttgtacgtggccgcatctcgtgcacgcaaacgcgtgatcatgtttgggcatctggctcagaaattcggaggcatcatttaagaggcagctatggcagagacctattcattcgggaaggcagcaggagctattcgtgatgccctcatggacaagctcagagagccagaggaaagacgctggcagctccagatagacaagctggttgatgccaactttgcttgtgatccaagtaacagcatgaaggggttcatgtactccgggcaacgctatattcatagccaagctcgaaccaagtacaagagctacccgatgctgtggcacacgctctggggtgagatggatgcgatactgttggatcaatccatgcatgacctgaaattttctcaggttggtcagatgatctatcgactgctttacgagtgcaacgatcgaatcgaagctcgtaataaccttccggagtgtctagttcagttgctgaacgaagatctgagaaacacgcctagaaagtttgtggaggggtacatactccgtgaaggctgtgattggcgtgcacagaaacagtttgaagagttgcttcccaccatggaggcaatggttgtcgctcgcatgatcatgtgagccaaggaggataaatgcgtcacctcgtgttcgaacagaacgatcagtacccggtgtgcctcttggttcaacagatcaatgaggacgccatccgaaaagagtacttggatccgcatccggacatcagccctgctgatgttctcgtactcgatctgcacacttatccggggaagaagaaagtacccaaggctgagatcgtgcagtacatcacggaaatgctcgtgcctgtgtggcaagagcagaagtgccagtacatcatgtgttccgatccggagtacttcaaggcactaacgggggaggcaaaagcggatgccaacctcgggtacgctctcccgtgcgtatacatggaagatgtaaaagtggtctacatcccgaactacaaggcagtcttctacgatcctgaaaaggttcgcagcaagatcaccattgcagtcgagacgttgatcgcatcagcaagggggcagtatcaagcccccggtgtcgggattatccagcaggcccactacccgaagactctgggagagattcaggagtggctccggaaactgttggacatgaatgttccgcttaccgtagacattgaggcatttagccttaaacactacgatgcgggcatcggcacgatcaccttctgttggagcaagtctgaaggcatcgccttccccgtggattatgtacctatccacggtgctgcttcagcaccctatggcagacaagtgcgcaacgaagagattcgctcgcttctgcgttggttcttcgaagagtgcagccaacgcatgatctaccataacatcgcgtacgacgtgtatgccctcatcttccagctttacatggacgatatcactgacaacgtgggactgcttcgcgggttgtccgtcatgttgaagaactgggattgcacgaagttgatcacctatctggcgaccaacagttgcagtggaaataagctcgggttgaaagaccagagccaagagtacacgggcaactatgcgcaagaggacatacacgacatcacgctgatccctctggatcagcttctccagtacaacctcattgatggactggcaacatggtttgtctacgagaaacactggggtacgctcgtcgctgacgatcaggaggatgtatatctgaatgtatttaagcccgcgaccaccgacatcatccaaatgcagttgaccggcatgccgatcaatatgccacgggtgcttgaggtcgaagtggctttgacggcagtgatgaatgatgcactcgctcgtatgaaccaaaccgaggcaatgcagaagtttgcataccggttgaaggaaaagtacatcgagaagaagcacaaggagtggaaggtgaagcggatcactctggcagaggtaccggacgatgtggtcttcaatcctcgttccccgctccaagtccaagacttgctttacggcatgttaggactgcccgtaatctcgttcacggatagtaagcaaccgtccgtagacgccgacacaatcgggaagctcaagaaccatacgaaagacgctgacacgttagcgttgttggatgcactcttggacttcgctgctgtggacaagattgtcgggagcttcatcccagcgatgaagaatgcccagcagggggcagatggatggtggtacttgtttggtaacttcaaccttggcggcactctgtccggccggttgtcgagtagtaatccgaacctacaaaaccttccatcgaatgttgccatggcaatcagtgatgcattgctggccatgtttgacatcctgaagcagtacacgaagaagggcaagctgagtctaggcaagctcatcaagtattgcttccaaccaccacccggttggctgtttgtcgggttggacttcgcgtcactggaggatcgcataagtgcgttgacaaccaaggatccgatgaagctgaaggtgtatatcgacggcttcgacggccattgtcttcgtgcttatgcgtacttcggggatcagatgccagacatcgatccgaatagcgtggagagcatcaacagtatcgcagagaagtacaaggtgctgagacaagacagtaaggcaccaacgttcgcactgacctatcaaggtacgtggagcaccctagtcaagaactgtggcttctccccggaagtcgctaaagccattgaggctcgttaccacgcactgtacgaagtatcggatcagtgggtgcagagcaagctggatgtggcagcgaaagtcgggtacgtcacagcagcctttggacttcgtgttcgtacgccactgctaaagcaagtcattcgtggaaccagtaagactccacatgaggcagaagcagaaggtagatcagcaggcaatgcgttgggccagagctggtgtctattgaactcccgtgctggctcggagttcatggggaaggtacgtgagtccgaattcaggaatgcaatccgaccatgtgcacagatccatgatgctcagtatttcatcatcaaggacgacattgctccggtgtcgttcacgaacaagcatctcgttgatgcagtgttctggcaggagcatccggatatccaacacgacaccgtaaaactcggtggcgaggtatccattttccaccctgattggagtgaagagatcgtgatcccgaactaccttcagggccaagagattttcgatttcattgacaaggaagcagcatgagcaacaaggcaaagttctaccacctgatcgcaacgaaggttctgttcgtgatcgagggccaagaagctgcacagtcggccgacctgaacgccatcctgtacacggaacagaacttcgtgaatgcgaagatgctggcacgtgctcagaagaacgtccagatccaactgcaccaagtgatcggtgaggacgtgaagtacaacgtcgcgaacgtgatcatcaccggcgtgtcgagcctcggctacatgacgccggaagagttcgcaggtgtggacgaagaaggtgatggtgctgtggccaaggtgttcgcatgagtaccgggccgaaagaaactcactcagtagtcagccctacgacgtacaaagacatcgctactggtgagtggttcatggacaatcaagacgaggtctacgtgaagttggacaatggcactgcccgatggatcggacagtatgtcgaaggcatcaagccatcatctttccacgacgacaaggtagtccgtcgtgtagtaggtgtccactacacggtgaaagcatgagcaaggaacgtaccggtggcaaggtgaactactaccttgcccaagtcacgaacccccagcgtgaagaacaagcaccgtatcaggctgagtgtgaggacatcatccaagcactcagcatgaccttcgacgaagggtgcgagttcaaggccatctggcgtactgctgcggcccgtatgggtctggagaaggacaccggaaaaggtttggtcgagaacgccttgtacgacgcacagaagcgcgtgcattacgctcaacgtagcgtgaaagagtacaaggctaaactggatgccaaggcaactcaaccgacaggttggatcacttgggaaagcaaggataataagtgtccggtgtttccgagtacaaaggtggaagtccgattccgatcgggtacttcgatcacttcaatcgcacctgaacagatcaactggagtcatctaaacggctccggtgatgtggttgcctatcgggtaatcgactgaaccaagggcaggaactctgagtgttttcttggctcttagcctgcccttcattcaatcgattaaggagaattaagtgatcacactagatgatctcaaggagcgtttgcactacgatccagagactggtgtgttctcgtttcgtaagacaggtaaggtagcaggtcacctaaacaaaaagagtggatacgtaacgatccgcttggatgggattagctactacgcacaccgtcttgcttggttctacatgaagggcgagtggcccgaacgtgcagaccatgagaacaacaaccgtagtgacaacaagtggacaaacattcgtgaagctactcaagcccagaacaatcgtaatgggcgaaagcgtgaagcaaacaagagtggctttaaaggtgtgtcatggaattcccaaagttccaagtggaaagcaagcatcacattcaatcgcaagcaagaaacgattggacagtctgattgcccaatcgaagcggcacgtatgtatgacgaacgtgcgatagaactacatggtgagtttgcaaaaaccaatgtagcacttggcttactaaaggagttgccatgagtatcacaaacgtaaacgatgtaccccttgcccttgcagtttgggcaatgtttgacgaatacgattacgtatccggtgtcgagaattacatctcagtgacttcgttgatgaagccccttcgacagatcgtcattccgccacgcattccccaagagaagcaggggcgtgctgacgtgatggacttcatcagcagtgcactgggtaaatccctgcacgactcggtggaaaaggcgtggacgaaaggctatgcagacaatctgcgagccttgggttacccagattctgtcatcgagaagatcgtagtgaacccggcacacacgccgagtgacgatcagatccccatctatctggagcaacgtgttctgaagaagttcatgggcttcactatcggtggcaagtacgacttgatcgctgacggtctgttgcaagacctgaagtcgactacggcctacacgtggttgtatggtggcaaggacgacgattaccaactgcaaggcagtctgtaccgctggttgaaccaagacaaggtgacagaggacttcatccgcatcaacttcatcttcaccgactggcagaagatgagtgcaaagcagaatcccaagtacccgcagaagcgtgtggagtacaaagacattccgctgatgacggtgcaggaaaccgagaactgggttcgagccaagctcaaccagatcgtcatgcacaagaacacgcctgagcctcagttgcctcgttgtaccgatgaggaactgtggcgttcggctccgcagtacaagtattacagcaaggtcgagactgctcaggcaggtggccgagcaacgaagaacttcgacaccttggtggaagccaagaccttctggatggttgagaagggtggtgtcggaaccgtcaaaaccgtaccgggtactgtaaaaagatgtgggtactgccccgcatttgaagggtgcactcaacgactggagtatgagttggaatgactgtgacactagaggtattgaaagagtaccttcactacgacagtgcctctggtatcttcacatgggtcaaagctgcatccagacgtgtgaaggtgggggatgtagcaggttctgcgcatagtgaaggatacgtctgtattgggctaaaaggcgaatcacactatgcccatattctggcatggtggatgtacacaggagttattccaagtgggcttattgaccacaggaatggcattaaacatgacaatcggataggtaatcttagagaagcgtcttacaccagtaacgcccaaaactgtggtaagcactcagacaatgagtgtgggtacaaaggtgttgctaaaaaccgtaaaaagttttctgcaaaaatccatgtaaatggcaagaaaatacaccttggaagttttgcaaccccagaagaagctgcaagagcttatgatgcaagagcattacaagaattcggggaatttgcaaaaaccaacgcgaatcttaaattactcaggagttagtatgattgatttgactggtgtgcaacaccatgcagccatcgaagagatggtcaacgttttgtgcagtaaaactcagaacacagatcgtggtttcttccgtgtggaagctgcttactttctgggtaagctggctgcgaatatgcgtgcagtgatcgtcaccaaggatcgtggcgaagtacctgtgaacatctatgccttggctctggcaacttcaggctttggcaaaggccattcggtgaacatcgtggaatcggacttcatgtccggcttcaagaagcgattcatggaggatagtttcggtgtcatcgctgaaaagcatttgtgggaaatggctcgtgctcgggctgctcgcaatgtctccgacgagcaagaagagtacgacacggttgaatccgagtatcgtcgtgcaggggcgtttgcctacacgttcgacagtggtactcccccggctgtaaaacagcttcgtcacaagcttcagcttgcgaactgtggtgcagtaaacttgcagatcgatgagatcggctcgaacctgtttaacagtcaggagttgctgaccctgttcttggagttgtatgaccaaggtctggtgaagcagaagctgacgaagaacacggccgagaacacccgaggcgaagagttggatggcaagacgccgaccaacctgctgctgttcgggactccgtcgaaactgctggacggtggccccaatgaagacatgttctacagcatgttggaaaccgggtatgcacgtcgctgtctgtttggctggggccaagtggacaagaaggctggcaacgtcatgtcggcagcagaggtctatgccaagctgatcgacaagagcaacgtgtcatcgctccagaagtggaatgccaagttccacaagcttgccgatccggcagtattcggctggaagatggaagtcgaagacgacgtagccatcaagctgatcgagtacaagatcgcatgcgaacaagcaagtgacaagctggcagagcacgaagagatcaagaaggcagagatgaaccatcgctacttcaaggctctcaagctcgctggtgcgtatgcgttcgtcgacgagagcaacaaggtcgagatggatcaccttctgtcagcaatcctgctggtggaggaatctggtgtggcattccagacgatcctcaatcgtgagaagacctacgtcaagctggcgaagtacatcgcagcagtgaacaccgatgtgactcacgcagacctgcacgaagcactgcccttctacaagacgggtaacgcagctcgaaacgagatgatgcagctcgcaactgcatggggctataagaagcacatcatcatcaagaagttcttcatcgatgggatcgagttcttccgtggagagtctctcaaagagactaacctcagcgagatgagtctgtcctatggcgatcactgggcgtataactatacgcacgaactggtgccgttcgatcagcttcagacgctctgtcaagcgagccaagaggacggatcagcgatgcactgggccaatcacggtttcaagaacggtcaccgtgccgaagagaacgtggtggccgggttcaacctgatcgtcatcgatgtggatggtggggtgtctctggacacggtgcacgacttgatgaaggactacaagtttgcaacgtacaccaccaaacgtcacggaattccggatgatcacggcattacccatgatcgtttccgtctcatcatcccgaccaactacgtgttgaatctggatcatgacgactacaaggagttcatgaacaacgtgatggagtggctcccgttcgcctcggacgagagtgccaatcaacgagccaagaagtgggaaacatatgcaggtggtacgttcttccagaacctcgaaggcgagttgctggatgtgctgcccttcattccgaagaccagcaagaacgaacagcatcgcgagaagatgtccgagatccagtcgatggacaacctcgaacgttggttcgctcaacgtattgcgtcgggtaaccgaaacaacaacatgctcaagttcgcaatggccctcgtggattccggtctcgatctgctgaccgtccaacgacaggtgctggccttcaacggtaagctcaacaatcctctcacggaacaagagttgaacagcacggtgctcgtctccgtggccaagaagtaccaccagtaatcaggtggctgggagcctttcttggttctcagcccatcaaggagaaacacatgagtgacacgcaagacgatgcaatcgatccggatttgatcaatgaccagttggtcttgatcgcaggtttttcgggcgagggcaagtcggcttcgttgcgaaacattcgcgaccagaaagactggttgtatctgaactgtgaggcaggcaagcgtttgccgttcgccaacaagttccagagctaccgtatcaccgatccgtatcaagtccatgacgcattcgatcatgcgacgaacgatgatacgtcgatcaagggcatcatcatcgattcatccacgttcttgatggacatgatggaatcgatgtacgtgctcggctcggccaacacccaagcagcatggggcagctatgcccaattcttcaaggtgctcatgcagcagaaggttacgttgttcggcaagcccgtcatcgtgatcgctcacttgctggacatctacgatgaagcgagccagacgatgaagaccggtgtgccggtcaagggttcgttgaagaacaacggcatcgaagcgtatttctcgacagtggttcatgccaagaaggtcacgttgaaggaactcgacaagaccgcaaacggcatgctggaaatcacggaagacgagcaggaactcggctacaagcacgtgttccagacccgcaacaccaagggaacggtcggctcccgtattcgcagtccgatgggcatgttctccaaggcggagacctacgtcgacaacgacgcccaaaagctgctcgaccacctcaccgcgtactacgcgtaactttcaccacgcagtaaaaaggaattcacaacatgttcggtaatctcaattcgcaaggcacggaagcagtcaaggactccctcggtggtggtggtttcgtcatcgacacggatgcgtacccgctcacgatcaaggcactctacggtggcaagtcgaagcacggttcgctgttcgtccaactgatcgcgacgaaggaagacggcaaggagtacaaggaaacgatctacgtcacgaaccagaagggcgagaacttctacacgaaggacaacaagaagtatctgctgccgggctacattctcgtcaacgatctgtgcaaggtcgtggcagacaaggaactccacgagatggatcaggaagacaaggtcttcaaggtctacgacgctgacgccaagggcgaagtgccgaagaacgttccgatgctcgtcgaagcagtcggtgccaagttcggtgcgctgatccagcgttacaaggagttcaagcaggtcaagaacgaattgaccggtgcatacgaagacacggcagacacccgtgagggcaacaacatcgaaaaggtgttcgacctcgattcgaagtacacggttgtcgaagcaacgaccggtgctgagtcggccacgttcgtcgatgcatggctcgaagccaagaagggcaaggtctacgacaagaccgcaggcaagacgccgaaggcaggtgcaggcaacaacggtgccccgccgaaggcaggacaagcagcaggtggcaatgcacgtccgtcgctgttcaagaagtcgtaatgcctgaacgctggcaacccatagccgggttcgctggctatgaggtatccaacttggggaacgtgagaacgttccgtcccaagaacggtcgtggacctttgaaagaagagtcacgacctgtccgacaagataaagcaaaaggtaaggaatacttccgagtatctcttagcgatggaaatggacacagcatgcacaggccagttcatcaactggttctggaagcgttcagtgggccaagaccatcacctgagcacgatgcttgtcatcgagacggaatacatacgaacaacgtcagcacgaatctgtattggggaaccaagcaggagaatgctgacgatcaaatccgtcatggcacccaagtgaaaggcgaacaagtaggcttggccgttcttacagacgatcaggtgcgagagattaaggaagcattgcccacgtggaaacgtggtatgggtcgttacttcgcagaaaaattcggcgtgggtgacacagcaatatctgaaatcaaacgcggcaatacgtggagacacctatgatcgtatgtggcctcgatccaagtttacgaaattgggggatagcctatggttggctagacctcaataccggcgagttaacagaccttcacttggatgtgattcaggtgaaggacgacacttccaagcaggttcggaagaactcgaaggatctccatatcgcccaatctctgtttgagggcgtacttccttatgccaaggaagcgaagttcatcttcgtggaagttccgattggctcccagtcagcgagagcgatggcatcgtatggcatttgtgttggtgtccttggttccttgcgtgcaagcggctacacgctcgttgaggtaactcccagcgaggtgaagatggctttcaccggtgataaggaagcatcgaagcacaagatgattacggcagctatgtcgtactacccggacacaaactggcccatgcaattgcagaagggtgtgaatcgggtcatcgagtccaaggcagagcacatggctgatgccatcgcctccatccatgcaggggtgcaaacacctctgttcacccagaccttgcaggtcttgaaaggaatgtgatccatgcagatcatctacaacgcagcagaactcgcagcaatcgtggagcgtaaccttcgcatgaagttcggtgtcccggacggtcaaggcgaaggcgatatccatgtccaattcgcgatcgtcgatggcgaaacgaaagcattcgtcggtgtcggtgagttcccgaccgtgccgcagaacacgctggcaccgtgggaagatcgtacccatgtgtcgatcggtgtcgatcacggcaagtcgatgtcggagacggtggccagcctcagcacgtcgctgagtacgactgaaccgcaacgtcgtacccgtcgaaccaaggccgagatggaagctttccgccagcaacaacaggcggaagctgaagcgaagcaggccgaaacgggaaacgaatccgaggtttcgaagagtggcgacgacgcctcgaagcaatcccaagccaccgatgcgaaccagcagcaggaagctggatccgaacaggctggatccgatggtgagcaggtgaagggtgacgcacaggagcagccaacgtataccccggccgctgaagtgccgatcgctgatccgaacgacacgcctccggcacaaccggatgaaggttcgacgggaaacgcccccgtggaatcgacttcgcagcaagaaggcacgcaagtaccgaccgctccgcgagtctcgctgttcgccaagaacaagcccgacgcgtcgacgactggtacggctggtgcccaagtttcgggtgacgcaccgcctgcatcgccccgtccaagcctgttcgcaggtcttaacaagaacgctggctaaatggtaagagccctactttgtggggctttgctgttggtcgttgtaagcgtgattgcgacggccatcaccatcatagccccgtacgtagcgatcgttgccgtactggctgtcctgatttggtggctaggtagcacacctgagaagtaacgaagccccatccgtaattggatggggctttttaaatgttagttaatgacgcttgcccacggattcagcatcggtgcatggaacaactggccggggccagctgcgtaaccaagagtgccattggccatcttggagaagaagttgtcaccagtcggcagaccaacaccaccgaacacatggggcaccggaaccaaggaagccagaagcgtatgcactggattgttacggatcatgctgactgcttgcttcgtcgagcggagcttgaagttcatgaaccagagcaggcccatttgctccaagtacccacggttacgtcccggcaggcgatcgtagttaacgaactcttccgagatttggcccagagctttctctttcgaccatcccttcttctgcgtcaggtgatcgaacaagatggctttaccgaggaaatcgccgtactcaaccatcttctggaggcccgtgtacagggcagtatccttggagacgattgcgtacttaccgacagtcttagcaccttccggaaggcgatcaaccagaccttccacatacgaacgccatttacccgatgccagctttacatcatccggcatcatatccgtagcatgcgagatacccgagaactcaccagcttcgatcaggggagcgatcgacatacgcttgaagctgtcgttgatcgattggatctcagcttgcagcttggtgcgttggttacggtcagtagcagcaaccagttcagcctctgcctcaacgatacgcgtacgacccttcacgtattcgttgatctcattcgtcttagccgggatagcaccgatggcagtcttgaacggaacaccacggccaaccagttgaagaatgttcgacatagcgttggcgaccggtacgatgacagacttgacaacgatcgtcgacttagcttcaccgaccacgttcttcacaaacttctcggcattggtgaacaccttgtacgcatcaagaccaatcgcaccgatgagagcacgcttgacagtatcttgcgtctcttggctccaacgcgagttgccagtccatgcgtcaccaaccgacgcttggcgatagccaagtgcatcgttcagcatgtcacggcgaaccttgaagccgtccgggaagcgatcagcgatcagggtacgcatctcacccgagatcagctttgcagcatcacggagaaccggatccttggacgagaacacatcttcgtactggttctcgttgcgtgagtctgccttgatgtcagcttcgtacatgtcagccagcttgccgatcagagcttcgttatataccttggctcggacttcttcaacttgacgaccagcccactggcccatgctcttggcaaggttacgttccgggttcagcttgctcatcatttccggatcgatggaacgctcgtatgcaaccaccgtgccgttttccgagtagaccgggagcagaggctctttggtttgaccttcgttggcgtaaccacgggtgatcgcagcaacctcgttgcgatccgtgatacggccagccagcagttgagtcgagtaaccggtgacagcatcaacacccgatgccgtgctacggacgttctggaacagaccttggtcgaatgctgcacgaccctgcaccggtgcgaagtagtagcccttcttctgcaccacgtcagccgacgaacccttgtacgaaccaatacgcttatggctcatctcagaccacttcgtatgttcgctgtccggtgcaacttgcagcgacagtccttgttgaccattctccggcgaccaacccttgtagtggttgatcagagcagccgacgtttctgcgacacggatgctctcgtctttacgcagacccttcaggtagttcagggagaagtccataccatccgactccgaaccaaccagcgaagcgagatcagtcttagccgatgccgattgctgatcaagtgcgtagagcgtaaccagatggtcgatctcacgttgagccgcacgatccgaaaccttcaggttcttcgtcttctcaccgaacatcttcgagatggcgtaggagttacgaagcagattgtgaccagcttcacccgtcaccatgaacttagcaagctgttgcatcttcgcttgcttagcaccccagttagccgggtcatgtgcttggatcgatgcctcaaggttgttgatgcgtgcatcgatctcagcttgggagccaagaagcttacgcacgtcttcaaccgagttgccgtcgagcaacgaagcgatgtccgtcttggccatgccttcatgcagcgtcttccattcaccttccgtcaactcacgggtgaacttcgaagcgatgatgtttggcacttgctcacggtagatctggcgagtcgcttggatgttcgacttaaccagcttgatcagatcgatcacgctggagttggactccgtacgaccaaccaagtcattcatgatggagcgcagaccgtcatgaagcttcgtcgtgttcaggtgtgcattcagtccttcagcaaggatcttgccacgttgttccgtagcaaggccgagcgtcagacgaccaaccttgtagaccgtcttggcaaggttgctgttcgactgcgattccagttgaccgagcttgtcgatacccgcttccgagtattccgagatcatctcaaccagtcgatcgttcgcgttgtcgaggaacgaatgtgccttgtccgacgagttctggatgaacgagttttcgtcctgcgtgatgtccatcaggcgatcatgcagtgcatccatagcagccgggacagtacgagcgtccttggttcccgtcaggcgacgcgacagtgcatccatcacaccgtaaccgatgttctcgatcaggcgatccgggctacgggtagcatccgacttcttggaacccggcataccgatcttagccagagctttacgagcatccgggttcgtcagagccaagcccataaacactgagaggttcgtgctgcgacccgatgcgtccgtctgaccaaccgtacggccaaggagtgcattgaacttggcattggcaggcgtgaacaggttcgtatgctgtgagtcttccgggttgtctaggaagtgctcaaccgtcaggttcttcgccacgtgagcgaaatactcttgagccttggccatcacgctcggatcgaagttggcttcagtagccatagcagcaacgatgcgacggaacgtgccagcttcttgcatagtcatcgggaacactgcttccacttcgttcgagactgccacagccttcgccactgcctcacgaacgttgccctttgcaaccacttgcaggggagcgttcttcacgtagtccgtgatcttgcgggtgaacgtgttacgtacttccgtgagacggtcatccgtaccgaacacacgtgactggaacagcgtcgactgagcagcttgagagttcagagtcggctccgagttcatcaggatggccgagttgaacagcaggtgcgagaacatgtccgtacccggcttcggttccggcagagccttccgaccccacaccagttgtttgatcgcagcgatagcagccttcgccacagcgaccagacccttggcctcggtcttacccgtcacagcagccagttccttgttcgacagaccccatgccatgaactcgttcagaccagcagccttacgggtgttcacgtcatatgccgggttgttcaatgcagcttcgatcgatgcttgagcattacggaacgactcatacgtcttggaatccgtcgagaacgactcttccatcccacggaattgatccatgagagtttcgagattgccgatggcttggctcactaccggcgacgactgaccgttgtagtgcgattgcaccttatcgaacgtggcagcatgcaccatttcgtgcagtaggttctctgccgtaccttccgtcgagacgatcaccttgttggccgtatccgtaaagcctgccgtgttctggtcgagctttgcagccagttccggcgagtaggcacgaatctgttccggcgtaccgaccagcaccttgtaacccttggcagcttcactgcgttggatttcacggagcaggttcgtttggatcgtgtcgaggttgttcgacttggcgagttgacccagtgccgtgtacgaaagcacacgaacacccgacttgtcttcacgaccgaggttcgagagatccatcttctcaaccggagcagcagcttctttcttgctgtcgagcttcgcacgttcttcagcgtagatctcgttcaggcgggcaagcttctgttctgccgtaccaaccagcgattccttggacgtgacacggtacggagcaccagcaccagccatctgatccatggagatattcagacgatcgatagtcttgtgacgtgcttcagcatccttccagcccaagtccaattcgttctgaagcagagtcagttgagcatgcagcatctcaggcgacggcgtttccttcgtaccgaggccattaagggcttggttcagttggttgtacatgtccgaaccttccacgatcatgtccggcgtcatgttgtccatgaacttgctgaaggagtcatacacagccttcagcgggttgccttgccacgaatcgtagacagccttgttcgccacttccgaaccctgtcggatttgatccaaggggaagtgcacaccgtcgaagattgggagcgtacccttcggcatcttcggatcgttgtacatgttctggatcatcttgccatcacccataccgatgttcaggaacgggataccagccacaccactgtcagcagggccatagacgaaacccggagtacggaaacgatcatcgagacccttaccgagaaccgacgactgcacttccgagttctgagaaccggagatgaagaagcgttggccgttcgagtcgatcatcggagcgtacttgtccagcgacttgagcacttcagcttgttcgttctggctcaggaattccgtcttcttacggccagcagcaaccacttcatccagcttggtgttgatggcttgcttgaagagttcaccgaggacgatcgactgagcttgcgtagccttgcgaaccatctctgccgagcccatgaccgagtcacccaccgtcgtgttgatggcagcacgaagcggatcgacgaacagcttacgcatgttcttcgtcatgttggctttctggttcttgccgaacgtaaaggtttcaaagttggtgatcggattgtgctccgatgcttccttcttaccgtaccagcccttcttgccctgctggagcgtgttctcgctcagatagttcactgcatcgttgaatgcagcccacttggcttcaccaccttcaccgaacatggcttcagccatcgacagcttggagtcgttcgcttgggcttgcagggcttccgacatggcttcgtagatgttcgtgtggagagcttccaccatcttcgcagcaataccttctgcacccgaaccatagacggtaatcgtcagcgggttcttggcgataccacgggacagttccagcttgccatccttgatatcgtagtcggacatgaacatgtccatcagcgtcgagaagtggctattgaataccgagccttcgccttgcagcgtttgagccaagtcagccagattgcccttcagcacgtcagtcgttgcttggtacagatccttgctatccaccgtcgagcgatagacttgcgaattctggtcttccgtaccaccaaagaagaagccacccttggccaagttgtcgatctgaccttgcgtgaaggagccacgggacagcaggcccatagcgttgatcgggccattggtcataccgtcagcttccatgtatttcgtcgtcacgaatgcttgcggatcgttcgtttccagcgagcgtgcgtgttcgagcagtgcatggagatacaccggagcttgacccttaacagcgtcgatcaggacatcagccgagaagtcttggttgttgaggaacttacgcagttcttcgatagccggagcattcttcgccagttcagcagcgagcttgttctccattgcttcacgttgcatgttgtggatcttcacacccagagcttgagccaagccaagatcgaaggcatgacgatctgcatcatcagtcaggtcgagagtcgcacggttcggaagcaggacttcacgagcagtcttggctgcttgcgggccatacttgccaagttgttgcagacgaccaacacgcgacatgttgaaggcgtagttcaccggaacctgatcttcagggataccgttctcttcagcatagcgacggacttcagccatcgtttgatccagttgaaccagagcacccatagtggtgatgttctggccatcgagggtcgcacgatgattgacgttcaggggcgtattacgaatgtccccagcagcgaacatttccatcagacgatcacccagcgactcgtacagacgatagacattcgagtctgcgtagtacggggtctcttggttcgctttgatggctttcatatcctgtgccgtattctgcacagcttggttacgcatctgcgtcttggcaaccggcacttccgtgttgatgtacaccggacgttccgtcttcacaccaaccgagtcagcgatagcagacggcatctggttgatcgcattttccttgtcgatcgagttcaccgtaatacggacgaagtccttcgggccaccatcaaccgacaggttgatcgtgttcacttcgatgtgaccggtcgcttccatagccgacaggagttcagcagccatcgacaagggaatacccttagtgtagccatccggggcagcattgttcgtttgcagaccccagaaggtcggcagcagttgagccaaggactgagtagcttcgagagccgacataccagcagacatctggttgaacagctcatccgacacatcggcttccttcatgccgagcatctgagcaacatcgctacggtcaacgatctgaccgtacaggttcgagttggcgaaccagtgcatagcagccagcgccgagttctggatgatctccgggttgtacgtcagcttgccaccgttctcttcaacgaggttcagaagcttgccatctgcccacgtatgcacgtcttcgccttgcataaagcgtgcaccaaccttgttcttcttcaggaagttggcgagttgggtgttcagttggtcaacaaccagaccagcgtcatccagcagacgaccgaattccttagccacttccttcggaagcttagccttcagatcagcacgtaccgtatcgatcggagcttccgtaccgatggtcgtgctcttggcttccttcggcagacggaatgcttcaacgaacttgttgccaccttggggaactacgagacccggatagacgtgctggatcgaatccttcggagcagccggatcagttgccttgggatcggacgtaacttcagaagcttgttcaacaaccttcggagcagtttccgaggttgtagcaggcgtaacatcttgttgcgaatcagatgcttccgacttacgtgcgacagtagtcttggtatccttcggggaaatcgtttcttccttaaccggtgcggcttcttgctgcacaccagacttgaacgactccacgacttgcttggcattgccagcagtcagacgagcgtccaagggagcctgagccatatgtttgccaccaagttgcgggaaagcatccaccagcgagttgtgaatatccgtaagcgtctgagcctctacgccgactcgctgagccagagcaaccgaacgctcgttgttcgtattcacaaacatgccattacgggacttcacccaaccacggtcggcactgctggacaaagcctcgtagtgcaccgtcgagttcacacccggaacggccgtgtcgagatgctcattgatcgccccgactttgttctgcatgtgttggacgaacttcgaaaagtccgcaagctgggccgaggcaccctcgatatcacctgcacgtacagcatcgcgaatgatacgagcatgttgcaaggctgagtactgacccggcccacggccgtcatcagtcttgatttgcttgcccactacgtcttccggcttcaggccgagacgagcagtttcctcatcctgtaccttggctccattcagcagagcagcagccgtacggagagcattgatctgggcatccgacaggttcagcttaccgtccttgttctgcttcaggatcgtagccacatcatccgggcttgccttcaggggagcgttctcagccacagcaatgtggttgttgatgtttgcatcacccagagtcgtaccggcctgaccatccgtaaccggcttcaggttatcagccaccttctccgtgagcgtctcagcagttcgttgagcacgttggatagccggggattcgttcagggatgagataacatcagccacaccgtgtgccatctgcacagctgggtgatctgccggaagaccttccagagccttgggattcgtctgaacgaattcgctgataccgttcagcgtctggttcaggattgcacccacatgggcacgttcctcttccgagttcttcgtgtcgttgacaaactggctcatcttctggagagcgtcgacttggttcgatgcaccatccgtagcacgagacagaacatccgaaccagccacagaagcggggtcgaattggaatgcaccagccagagcatccacgtgctcttgtgcagcttgcttctgttccgggctgagttccggtgcagcttggatatcagccttggccagatcagcttgaaccggcacttcttgtgcaacggtagcagcagcttgcgagacgttcgtatccgagacaggcgactgttctttagcagcagcaatcttcttgtcgccacgagcagccagccagttggtcagagcagacgtgccagcaacgagaggcttggtagctgcaatggttgcagccttgcccaccgtagcagccttgccagccagttgtgcaccaacacccggagcttgggtgagaccagcagaaccaagaccaccgacgagaccctgaacagcctgatcaccaacaccttctgcgaggttctggttcacgttcgaagtgagctgcttagccttgttctgggacagttgaccagtgatcgactgaagcgtttcttctgcaccttccttcaggacgttacgacctgcttcacgcagcgacggaaccgagaacggatgggcttcgaacttcgacgcgatgacacccgtagcagcagcgagaggggtctgaatagcagcagccagaagaccagcacggttagcgacaaccgagcgtgcttcttcttgcgttgcaccaccagccacgagttcgttgaacatcgggctgttcttcttgagatcagcaaaggaagcaccttggatgtcgccagcattcgactggtacgcaccaccaccttcttgcagaccgatagcagcaggcatcgtccacttcgtaccgatgaagttggcgacacgtgcagccgaccacgtacccatggatgcgtcgatagccgctgctgcttggataccggcttgcgttgccttcggcactacagccgatccagcagaagccaaaccacgagaaagagggccagcagtaaggatagaaccaacaccttgagcagcaatatctccgacaagctcagggctatcggccaagcgaccggcagaatcaagagcatcacgaccaacacgcttgagaccagccatgaagccagaatcaccagcagctttatcagcctgttgttgggcttctcgtacaggggtttggagtgcactgaggttctcgctgatgttgcgagcagccttcatctggggagactgggtggactgcccgaattcattcaggtcttgcaggctctttgccatagcagggccagcacccgtcatcggaagagtcaagccagcaccgagtgcggcaagtccaccgacagagttcgcaagaccgaggccaatacccgtaaccgtatcagcagccacttggctacccgaacgggggggttgggattggtcttgacggagtgcacgtgcaccggcagcacggtcagccatcatctgggtggcttgattaccgtacttggcatagagggcgagaggatccatggtggcagcatcacgctgacgttcatccatggcagaaagaccaacaccagtctgaccagcaagagccgtctgatagttctggggagtgttttggccttggttcatcacggccattttctgatcagcagcattcgcaacatctaccttcttttgctctgccgtcagtgcgacgttagcggtagaagggaggtagctgctggcgcttgctggttgcggtgccgcacctgcgtactgggccaacaggtcgagtgcattcgacattgctgtttccttattagaagggagtttgaggaaggactatagctgaagtcctagaaagaagaaagcccctcactaggaggggcttcgattttacttcttattaggctggaagttcatcggcaacatcgccggattctgctgagtctgagccagtgacagagcaagtgcctgctgtgctgcttgcaggtttgcctgatacgtaggcagtcgatcacgcagggacgggtcgaactgagcacgacgagtagcttctgcaagctgagcagacgcttggttgaaggcatctcgtgcaccctgtacagcagcttgctgatccttgataccttggttgtccaaggtctgaacgatcgtatcaccggacttaaggccagcgatatcggccttgatgccttccatgttgaaacgcttgccaccggcaaggttcgacgtgtgttcttgggaagtccagtcacccgagaagaactgaccgaggtttcgagcagcacgacggaagccaatgccattcggacgccacgacgagttgttgtagctgtcgtcagcttgatcgatgttacgagccaagacagcagcagccgtagcagcgttgacgttaccatcacgcatgatccgattgagctgaccgacaatcacaccttccggcacattctggagcacagtcttacgaagctcctgtgcagcttccacattcgaggcagtcgagggcagagtggtggtgagatcaggagacacaccttgggcctgattctgcgacagacgggactgcatgagagcaccggcagcgttggcagcagtacccaagtcacgttggttggcacgaatctccgggagactctgaccaacttcaccaccagcgatgacgttacgcatctgttcccacggaacatccttgtatgcacccggagtcgcattcggaagagatgcccacgtcgacttgaggttgccacccttgttgtcgtcgaagatagccttggccagcttgtcttggttctccggggtcatctgggtgttcttccagttcgcaccaaagaccttcgggccatagttctccatcgtaccggccgtgatctggaaagcacccatggccgagctacccttgtccgtacctgccaagccgaggcttgcattgttacgggtattcgggatgagggtgttcttcccgaaattgatggcatcacccatgctcatcgaagtcaggggctgctgaggcgtgccgaatgccccattgccgatcatggtgtcgaaagcattgcctcgtgcaccacctgcacctgcaccacccggagcaacgccggacgaactcggtgcaccggcaaacagagcacccccagcttgggccgtaccaatcgggccaatcgtaccgggaaacatctggttggcacgaccgagcaaagcttgcaaagcacccggagacaggttgggagccaagcgagcaataccagcacgcatgtcatcggccgtaagcgaagtacgggacagttcgttgaaagcagcagcagcagcttggttgtcgttgtcctgacggtaagccactgcattgtcgaagccggtacgttggttgccaaggtcaccacgaaccagaccctgattgttcgatgcaatctgagcctgttgatccggagtcaaccccccgagaacgccttggttctgcgcatatgcgctctgcaagccaacacgatcgcctcgttgggcagcaccaagcagttctgcaactgccgggcctgctgcatccgtggcagcgttcagcttctgctgacggccaaagttgtacgcacccttgtccagttcctgttggttcgttgcttggttgatcagttgaccaacacgggaaccaagagcagcagcaccatcagccgagatacgcgaggtgttgatgccattgaacaacgaaccgtccaccagagcattccggtagtcgttcggattctggaaacggagagcagccaactgtgcagcagcgtcagcctgtcgagtttgctcacggccaaaggcaccgattgcatccgacagaccactagctgcacctgcaaactggttggcagctacacgggaagcgtcaacggccgtaccgagattcggtgcgtctacgttacgccaagtcaaagccatagcactttctccttatcgcgagatacggttacgatcgacatacgaggcgatctggtcagcagtctggccttccgctgcaccacgtgcacgtgctcgatcttcaagagccgtattgtacgacttgatcgagttgttcaggttcgtattggtgatgcccttcgtgaaatcgaactgatcacgagccagcttctgagcttggaaaccaccccagagattagccagagagccaatggcacccagaccaagctgaagggtaggcatgttcaggccgaggccaccaaagctaccggcaccattaccgagagcaccagtggccccacccagagggccaccacccagagtcggactggccatgccataggtattaagcgaaccgtacggtactgcaccaacgggggcaccacccccaaaattgaaggaattggcacccaacccccactggttgttttgaccgaagtcgtacataaaatctccgtttaagtaatcccttggctcggatccaaggaggttgaagcaaaatcgctgatcatcgcatgggagagttctgcgatatcagatccggtgagaagcgtacgggacaagaaagtctccggcttctcagggtacatctggtcatgcgtaggatcgacaagctgaagcgggttgatgttggcattggcataaccaatattctgtccccacaaacgctgaatctcttccatctgcgtattgtacgactccatgagtcgagcagtgttatttaccatctccatggcactgatctgctgatactctccgatgcccttggcagtagcactggtaagggaaagaagactcatgggactggtgagtgcagccataccaccagccagagaaccagtggtcatgtactgctggcccatgacgacagcgataacgccagcgatagcaccgatcactgcaccaagcttgtcaccgaacaccatggtggaaacttgcccgatgatcttcatgatcaacatcgcagcgatggcattaacagcagcaccaaccacaagagcagccacaccggtaagaccaagagcagcccctgtagccatggcagcagtggcgaaaggcgtaccagtaccccacgtgaagtacgtgataacgaccacgatcacgaccatgatgatctggaagaatccggactgccaccatttcttcttcttctcaacgtagcagttgaagaccaagaaacaagaggcagtgctcatctgggttgcatccttcagagtcatttggcgatacattgcctcgtgcaatgggatgatgaaacccgattcgtcactgtctgctagggcatccttagcggaagtgttgaccgaatgccccttgtaaatcatgttctggtgccatgcaccccagatatcgagacgctcatactgagtgtcgctgatctggcgactgagtcgaatatggctgatggactgtgtaccaacaccgatccatgggccaagagcaccaccaacaggaacaagctcatcctgtggactgatctcgtacatggtgaacgcgtcatctgggagtacatcccatttgtactcacccttacgcccgatagagccattgatcgtggtttccgagatgcccacccagaagatggtcatgtcgtagttcagtacacggttattatccgaggatactcgaatggacgatccgggaacattgggatatggcttgatctcaggacgtggtttgataggctgaccccagccaacaaacccaccagtcttcttccactccaagtagtctttcagcgattgtgtggctgcttcccagtcttgcttgaactgttcgtacaagccaaggttggctggactctgatccagaagaaccttgaagaagttataaatgtactgtcgggatgcattctccttaacgttcagtgctacgccataaacagcataggcgtagtcgatatccccaacttggggattcttcttgatctcgtccatcacgtcttggaacttgccaccagatcccttcttgaatgccttcttggcccaagggtagagactggcgtactgaggtgagtcgacgttgatgttgtcgatacggaaaggtacgaacggataaaacttacccatgttcgtgggacgagcgaacatcgagtccaatactgcattgccctgcccttgaccgtagatgaacacgttctgagggccatacgtattgaccaccgtagtctgcttgtccactcgataggatcgagtgacctgcaaggtctgggtggttgtcgtagtggtagtggttttcttggttccatccggaagagtttcttccttcgtctcaaccacaggggaaccaatgaccacattacccacctgatcctgatacatggtctcaaccagcatcgacgtatagttgggatcgatctgggtgttggtctgacgaccgtacacaccatgcgtttccgaatacgaatacggagtctgagtagtgtccgtagtgtgctcatccggtcgaccatcgctatacgtgatcttggtatccaccaccttgtccagattgccagacttggagatattcgaagtagacacgacattccatcccgagataggagggaacggatcacctgaaccaaggatgacagtattgcccggaatcagtgggccaacctgatcagggccagtctccatgtacgtgacatagaggtatcggatcgactggtcgtaaccaacaggtgtgaacgtgtccttggtcttgtccgagtacgtaatggtgacaatgccagtagcatcgtcgtactcagcagaccatgtatcgctcaccttattgggtcgattctcacgaagccacttgtccacccagatgttgaggttagccaacccaatcgtagctgactggatagttgccttgttaccgggcgagacaggaagttgggttgcgataacagtctggttgagcgtatcccctgacagcacagtgccagtaaccaaaccgatgatgttgttgtatccggagtagtcagcccatcgtgcaaatgaccggaacttgataccggggccattcagatatgagccaacgatcgaatcacccatggaagaggattgaccgagaacatccccaactaccgtctgacgaaggtagttggggcgtttgttctcatctccggcgaggttgtataccacactcgaaacaacgatgacggaaccaccaccgaagagacccatggttagacgaacccgttattgaccttgatggtctggaacaccttgtccaccgaggcattctcaaatgccgggggaggcacaagaccctcgtcgaccgtcttctgcgtgatccaagcatccgagaagatcttggcagctttgacttccgagtcacgcttgtagctggtgatctgctgatcgtacagatccttctgcttaccgagtacaccacgaactgcctgaccatccgtacggacatcgagggtctgtgcacgttgagcttccgtttgctcttgggtcatgaccagttgcttatccagcagttggctatttttcggaaggatgttatccacctgaaatttaccagcaccgaagttggtcgattccgtagccagcttggccttggtgagagcgtagttggccttcgaagtcagggcatcgaactgcttgccgatgagttcgaacaacgcagtcttcaggttcacttgtgcagtgacagcctgaatctgagccagttggccttgccagaatgcaccatcacgttgggtgaggtactggacggcattctgcatacacgcatccgtgagtgcagtgtatgccttcgtgtactcggcaccggtgatacgccccttggaaaactcttccttcaggtgacaggacaccgactccatgagtgcatcgaacgtaccatcacccttgggatgacgttgcgtgagcgactcattggtgagaggtttcaggcccgagtacatgggcgagttgggatcgtacttgatctggaatgccgggtcgttgatatcgacggtggggaatgagacatcctcacctgccagcagagcgtcatacatctgcttggcttcaagatctgcaccgtacatcgtgctcatatatttctccttggttcagaatggattaagcccgtggagtctacactctcacgggctttttagatacttcctagtggaagcttagtccaccgatccggcagcgagttgggccgtagccagttgcttgatctcttccggagtcagcggatcgagcacttcgaggctgtattccgaaacccagcgttgttcggtgtgggtcgtaccggtacggcgatctttacggagcttgatgtcgaggaacttgcgatccttcatcatctggtacagacagtacgggacatgccaaccttcgtccgtcacttcaccgtaggggatgtacttgcggatcgtgccgatgtattcgttcgccaccgtgaggatctcacccgggagatccttcttcttggggtcgaggttcgtgatacgcagacggatgagcttcgtagcttcatcgatcagttgctggcggaatgacttctgaccagcaggcttggcacctgcaacatcaccagccagtgggttcagttcagcaggttccttgtcttctgctttcggggcttgtgcttcagcaatgcgagccttcagagcttcgacaccgatgttgttgctgaacttgacgccgagcagcgtggcacgttgcttcagcagatcgagttccgagaattcttggacttgttccagcgattggccgtcttggccattgttgagatcttcgctcatttctgtaccttgttgggtgagggtgaaccaaggggaagattatccccttggatccttagtcactaagtccgattagatcggggcgacggtcttgaccagagcgatacgctccggacgcttgatcagaataccgtagtaccacttgatcgagctgaacccggtttcaccgtacgggtcgttacggtcagccgtagctgcacccggcatcttcgtcgtcaccgagaacttcaccgacttgccgtccgtctggaagccgatcgtggtgaacgagtcatcaccaatacacagcatcgggaacacgtcgtagtgttcttcgccacccttcgtcgaggtacggtagcccggattagctgccgtagctactgcaccgacacccgcccagtgcagcatttccggcacttggatgatgtggaacttgtcgatcgagccgatctcaccgttcagcaccgtaccagcagccgagtagtgttggatctcgatgaacgccttgttgccgaacagatccttcatttccttcagggccgggacgagttccgagccgacatacaggacacgcgttgcacccagaaccttcgtgtcgatcagacggctacccgtgatgatcgtcgtctgcgtcggagtacggttgtccgtcaggatctgatccagacgcatcaggttcttgtacgagaccatcgagacggcagtaccttcacccgtgatcgtggcatccgacgtagcagcacctgcgtacaggatcacgccagcagcagccagtaggtcacgttgcagaacagcttcggtcagttggactgcaccggtcatcagttcacgcgacaggtgatccatcaggccatcgtccgaatcgaagtccagagcttcttgagtgaattcggtgaagaaaccgaacttgtggatcgaaccctcacgttgcagacgggtgaacccaactcggttcactcggccaccgttttccgtcagcgtcggcagcttcgacgtgatcgtacccacgtccttgctagaaccatacaggttgccgttcgcgatcttcacgccgttggcatcgataccttggtcgttgatgttgcgatcgtccagcagcggcacgtattcgtacacgcggatcgacttgccgtagtgcttcggcatgttcgtggtcgacgcgaggggcatgaaatactgctccttacgtgccgtgatgatcgccttcttcagccagaagaaggtgagcatctgatccgagccattgccgtcaatgctcgatttttggccctcggccggtgcgttgtagttcaacatttggtctgtttccttttagacgcggttgttcatttgcttcaagaatgcatcgtcatccatatcgagatagttgacgatgggtttcgccttaacagcaacggcacgattgcccgatgctgcatttgccgtgtcactgttcgctaccttcgtaaccggcttcggtgcagctacagcgatgggttgcttggtggtcgtttgagccacggagggcttaacgatatcagcaaacttcccttcactctgcattgcgtcaccaactgccttatacgcttgcaggaacggagttccggcaggaatttgcccaagcaccgtgagtcgagtgatctcagtcgtaatgcgatcgtagatcccattactacgctgttcatcaaacactcgcagaagcgaggggtctttccagaggacatccttgctggcttgatcccacgtcgtgttgatcgtctgtaccgtttctacgccggactgggtggatacgagatcgtccagtgccgattgaaacgctacgtgctcatcactgacggcatgattgcccggcaggtaagtcgagtctttctctacatccagcgtcatcgggtcgatgttgctgtccttgagcagcttctggatcgcctcgggattcttcttatcgatatcgatcagataagaaagtttgccttcgtcgagcagaccgttctgttccagcatcatcagcaccttacgatggggctgaatgtcttgcatcttgcgagtgtagttggcacccatctgcatcaactggattgcctcgtctgcgttacgcagttcgatggtcttcccgtttgccttgaagggagcaagcaacttcttgtactcggcttcgtagtcgacggtcgcttcagtcgtcgtagcagccgccttactggcagctttctgctcggaattcccagcgttcgaatcgaggggcttcccgttggcatctactttcgattctgcgttatcgtcagcatttgccgcaaccttaccaccattcggatcatttttggaagcttcttctgcttcctgagccagacgggcagctttttcctcttccgtctcttccttgttggcagcttgctcagcagccagtcgatcggcttcttcctgatccagacgtgcttgttctgccgcagcagcttcagcagcttcacgtgcagcagcctcttcggcagcgatctcttcaggggtcttggtggggacttcgaacacgggagccgtgctcatgttttcgatctgctcgtccgacaagtcaaagatgtcggacggctgatccagtttaaggtcagccgtcgtcatcgtttactccgcttcgtcagtttcggtgttggcttctgcgagtcgggcctcttccagaccctcacgcagctcttcgagttgtgcaccacaggtttctgcttgcatcagttggatcacgatccaacgcttcagatgacccggtgcttggctcagagccagtgcatcagcacgttcacgatcggtcagggccgggttacctgcttcggcagcgtatcgtgcagcttccttggtcgagaactcgtcgatgatgaccttcttgaattccttgttttccaagagtttcgaaagggctgcaccgagggccacttgtgctttggcaccggccaactcgttctcgaacttctgcaccagactggtattgatcatttttgcaattccttaactgaggttgattgctgggttaagagcagggtcaagactcgggttaaattcctgagaaccaaggttcaaaggattcgtccccgctagtccccctgctgcatcacgcgagatagtagacgaaggtgcagaagaattcaagttaccgctaattttattaaaacccacggctgcttgaatatcaggagccttctcaccttccttggtcggggatgtaagagccttcgtaaccgcaaggttctggttaccttccgactgtgccttctgaagctccatctcacgagcatgcttcgtaccggtttcggtttccacgtactccagattcttgagatcgccagatgcaccttgcatcttagcttgagccaagaagagttcagcctgtgcctgattcttgccgatatccgattgaagcttctcgatctccagttgcttcttctgctgttcaagaggatccggctggggttggtaagtcttgagggcatgcgccagttcaggcatacgcttcagcttagcgatctcaacgaggatgatctgaaccagtccccaatcaccattcggccccatggtctgaagcatgaatgccagatcttgcgacttctggttatccacctcagcagtggagatatcagcgatcacttcgaagttgcctttcagatcctcacgagagacttctacgaactgttcgttggtcaccttgaccgtttctttctcggacagccagacagcattcatgctgatcagcttcatcgcgatctcagccatccccttagccatgcgacgaaggatggccatttctcgcttagaagatgcatcgagcaccccacgaataccagcagcaacatcaccatacgcatctccggaaacaccaccagcaaaggatttaacaccggtaagtgcttcagcctcttggttctgaaggttgagcatgagcatcacggattgtggcagttccgggtacttgtgctccatcaaaccaccggcagcaggattctggttggggttgaactcatagtcctgcccgttatcgaagcgacgcttgttcacagcatccagcatgcccttggcaataccagtctgagcattggccgacttgcccatcaggtcgatagcacctcgggtaactgcacccaagatcgcctgattgtcttccagcagttccgcatccggttcaccgtaaagctcacgcttgaccggcagatacgggatcaacacgaacggaagcttctcatccgggaacgggttcttctccagtcggatcatcacatcaccgatccaagtggcgacgatgggggtcagtgcaccattcccctcgatatcccagaagccccagtactcataagccacgaccttcttacgtgccttgtctttgaactggaacgagtcaggagtagtggtttcatgatccggttgagccaagggggtattaccttcccagttcacacggtcgagattcttgtaccgcttcttgtccttcttcagttcagaccagcttgtttcgaaggagatcaccacgaactgtgccttctcgaaatccccgttacatgaggggtcgatgaagacgttgtggggattcatgatctccagagacggctgattgaagagaatcttctcttcctgaatggtcgtggtctcaccagtagcttgagcgtaaacagggatctggttttcttccgtgtattccactgccatccgaacttcttccggcagttgatcgtatccacggggatttgagttcttcaactccacggcctgttggagttgctgaagctgatcttcagtctcgatctggtagtacgcgtacagaggcacttccttgggaagcatgatcgtctgacgattccaaccaagcttgatgatgcacgtaccatcatccacagtggaacgcaccatgtcgtcgatgaacttgaccttgttcagcttggttcggaattgccagttcagtacaagctcgttctgctgggcagcgtctacatctgcccacgtagccggactcaccttgaacagcttatccgagccaaggaaaggctcagtcagggcagagtaccgccactcagcctgacgacgaatcagcttcggctgtacttgcgatcggcccttgactgctttcggtttggcagctccccgtacattccggagatcgttccatcgttgaatgtgccccgtctgtacttgttgagaaggctgtgcctgctccaagtcgtgcttcaggtcttgaatgctcggttccttcccccagtccgtcaacttctgagggagcaaagtgctcggagcgagttgcagcttttcgtccattttggtcatccgtaaaaaggtttcggtcgttgatcagttgtttgccgactgcttgcaactgagagtcacgcagtctaagagtttccccgagttctcgtaccagacttctgccttcttcaagagatcggtcgagttcggctgtatggcttgcaagatctcggcaggaatcggatccgccttgggcttgacgttgatataccgaggctctggcttcagactgctgcaccctgagattgtactcactacgagcagcagcaaggtcaactgcatgggactcacgagctttccggagtgcactctcgatctgagttgtttcagcacgatggatttcctctttcacaacaagttgttcttgcaggtcttggatcttggcatgggtctcaatcttctcttggttccatgcagcttggacgctagtcttaccagcatccctgccaaagtagtaaccaagagcgaagaccgtaagggcaatgcccacgtagctgaacaccttcagtacggtctctttcatcatcgatccccttcacaagccttgtcgatgttggagtcgattcggttatcgatccagccgaccgtgaagtccgacatcttcagaccatcgtagtgaccggtctgcttggcattcaggagcttcagggtaagggtacatgccttgaacttccctcgctttttctgaagggcttggtatgcacctaccgttccggggccgactttaccatcgacctgaatttgaggaaagtccttaccaccacgggacagggagttaagactctcttggaaccaacgggaaggacgagcagggccgacatttacaccggagtcgaccagcttgtgcatgatcggaaccgagacagtctcgaaacggatgaaacccggatcgacgatgtacttctggacatagatgttggaagcatcctccttggtcagatccttcatggagccgatatagccagccgaacgtgcgacagacaccgtcaccccatgattggtctcaccacccggatccttcgggttgttcacgtatccaccttccactgcgaagatcgatgccaccatggcagcaacgacagcagagatggatgccttctgtttaagattgattgccattggaacctccgaacttgatgatccctcggatgatggcagcaccaaacagcacaatcgagattgacttggtgtgttggaaatccgctttcagttccggtggaattgcctgccacgcttgggtcaggatttcagggagagccagaacagctacccctgcgtaagaaagccaagtagcaggttccttgtgaacctgacgaaccttggacttcttctttaccggtttgacaaccattggatgatctcccctcgaatagtgaaaaggaacgagatgatccccccaaggatcgcccacgtccacttaccagccagtcctgcaccaactaccttatgcttgatggtgatgaattcttcgatggtaggggcttgttgcttcaagctaccctccacaacctgaactcggttatccatctgagttaccgtctggctcatctcttcaagcttctcgtactgaatcttacgcgaggctttcgcctcgctcagttcagtagcgatgaagttgaagcgttcttcaagacgtgcaagacgcactttttcgtcctcgttcatagccccgaccattggttaaagtaaagaaaacgggagcctaagctcccgtgcgaatagtacctcaagttacgttttgctacagatcaaatggtggacgggtcttcggcttctcgtaatccggaaccacagtgatgatgtcctcacgatctgtctcaagggccactcggctggagtggggcaggaagaagttcttgggttcttgtttccagtcagcgaatggaacaaaaccccagaacgagcgaactggggttttcatggttacaccgccgggtcgatcggatccggatcatcgaccgcatctttgtattccggaagggtacgcagatagtcgtacgcttgacggaaggggttggccccctcgatgtcgtacttgatcgagtacatctccgaggcgaaagcgttcggcgcaccagcacgaacatacttgcggatcgagaacgtgttgatcgaaccagccagacgaacttcctcgactcgaacgtatgcaccgtttaccgggatatcgcccggtgccgtcgtatttacgataagacccattttggtcacctcttagaaccattgcacattgaatcgtgcggtgttagtgaaactcaagggcactcccgagagttggttaatgcgtactgagagagtttgtcccggggttactgggatgactccagtgacaagggagcaattactagcaccactggacattttctcttcttcggctacaggcccaccatcaaggtagatacctacgtatcgttgcccagtgccattggattgccacgatccgttgagggtagcagatgcccacttaacattgggcggtacaacaatgagcatcttaccagcctcatcaaatgcttcaagacggtcgtactgtgaacccgaccagactacagtttgcggtacgccagactgaactgtgatgcctgacgagaggtaacagttcgtatgcgcaggttgtgtcccccacgtcatacacccatctgcattggcgaaatccaacgtatccccatagatagagatgtctgcaccagcgccaacaactacttgttgccgaacgctgaaccccggcagattacctgaacgacaccacgcaccgtgtccaaacgatgggtggtttgtgagactctttggctgaatcgtagtcaaaacttccatgcggccaacgcaagcatagaagaaatggttctgcgccagtgaacccgggtaatctgctgacaccggattcgtaccagtatccgatgcatgcaaggcgatagtaccgttccaacgacagttaaagaacgtattatcgtccgagttctcgatgtcaacgtgtcgactttgatagtctgagttgaaccattgacagttgtagaagatgcactggttcacgccacccttcaacccattaccgaacatcaacacagacttagctgtattgcccggaagcatagcagtggacacacagaagttgtcaaactcacacagtgagatagatgtgttagttcccgatccaccgtcatacaggtcgtgggtagtaacacccaataccagccccagctgggtgtgtcgaaccacagagctgtttacgtacttagctccacgtgtggccagaatcttcacaccgtgggttgctcgattattgcagtcaaacattaccccttcaatacccccaccctgaagtggtgcagctttaagacccggttcatctttaggggaaattaccagcatgaacgagggagtagttgcgccaacccactggagagtcgaacctgcgttagctacgatgttgtcccaagtgtcttggaacggctggttaccatcgattgccccaccgtatccacggataagaatatacggggaatcgacaataatggtctgggaaatcttgataacgcctttaggcaagttaatagtggcacccttaagacgtttagcctcagccactgcacgaataactgcacctgaccaatctgcttcaccagcgatgaagaatgaatccacataaagctggttatccagaattgcagcaacagtctggacttgtccattaggacgacgataaccaataagaccagcacctgctgaagaatgcaatgcatcaattgcctgcttgttctgactgatctgatccagatacccagacagatcgaagatgttacgccaagcagaatcaggctcgttcgtgtacttccactggatatgtgcatcagcagacactcgaatctccggggactgcccagtgatgtcgatgagcggaagcaagacctgccacgggcctttcgtaccatccttctcgatgtatcgccaatccagcgtcttgttggcgacgttctcctgaagctcaatgtccttgggacgaagctcgttcaggttctcggccagatacttgatgtgttgaagctgacggtaaacagcttccacaatcgggaagctgttgcccagtcgcttatcaacagacatcgaagcaccaccgaaggggttgtgtgctccacgttgattcatgtgttcctcacagttagacggatacggcaagtttgcctgtaccagtgccagcacttgcaaacttgatctgtgccgtgatggtagtggagttcgtgccaatcacagccatccaagcaataggcggaatgctaccagattcatatgagaaggtaagagatacatcctctcggttgggggttcgtacaagcttatgagagagggttaccgtctgtacagcagtagaggacaagtcaatagccccatacccaacaaacttgttggagaagttgtccgtgccatcgtaattgctgatatcagcaaccttgtacatacgagggcctaagcgccccaagttaccgaaagcagtggtaaccggtcgatccgaacgcagacgatagatggttccgatgtcggcattcacaccacccggaaggttgatgttcgtatcacagaaacgagccgacacatcaaggtcgttacgtacggtagtcgaagcttcttcgtacccaacttgcatggtgttggcatccctcttggatacaaccgtagccttgatcttgctggacgatgcttcgttcaggaagcccttgccaccaccagtgaatgcgttcgaatacccgtacacctgtgcagcgatgtcatggtcggaaccaacgagatggataccgagactaccgttctcaggagcatcacggaacacgtcgatcttggcctgcccaccgagtgtagaaatgatcttcacttggggattggctccagcgagagcatcattcgggccaccctttacagggccacgtcgattcgagtgagtctccagaaggttgatgttgtatcggacagcatcaccgatgaagacgttgccgtatgagttctcacccatcaggtagttggcccggaagcgaatcgacgggtttcctgccttacgttcgatgtgcaccgcatacccatactcattgtcgtacgagtggataaagcccacctcgcacgaacgaaggaggcggataccgtggatcggatcaccggggaacagcttagactgtttcgggccggtggcatcaaacgtatcaaaacgggaaccacccggccaaccaacgaagatgttgtccagatattgatcagttgggccgttgaagatgaacccttccatgcccgtttccagcacttcgatatcccggatctcggaaaacttcgagtccgtgatcgagtcgaaattggtgtaagaagggatagcagggaactcggtgtacagaccaatacctgctgtcttgccgatgaccagactcttcagtcgaagcatacggccatagatgcgaacaccgtaaccggtcttatcggtaggttgacctgagaacacggcaccattcacggtgcagttctcaacaccatacgagatggggcaaccatcagcagtacctgccggttgggttgcttgaatggcatcgaagttttccgtctccacaacgttgcctgcaaagcctgcttgagtgacgatctgggaagatgcagccccagagccaaccaaggtaacggtagatactagtttgataccagtgtataccttgaggattccgttgaaatgaacttcgccaatgccaagagacttagcagttttcattgctatattggcagcaaagtcagaatccccgtaccactgaagttggatgaccccactgaactgacggcggaaggtctttccggaattcgtagtcagaaacacgccaccatcatctgtcccggtagctcgaacgaagagaccaccagtaagcacgtcagtaacgatgagagcatctgcagtaccagtgtactgacgaagtgccgtgtaattagccacacgaggtgtggaggcagcaattgctgatgtagcagcattctcggcaagggtagctgaagcttgtgctgccgctgccgcatcaagcgcctcttgtgttgcagatccagtatcacctttcggccccttgaagaatgggtacaactggatcacattatcatcacacgacataaatacctcagttggttacgtgatcaactacgtcaatgaaggaggtagtcgtatgtacaacttccccattcgagtcgaccaccttgaaatcgatgtagtgtacttgcggacgccacttctccgtatctttaggaggtgcgtacacacgtacggtattggtctgtgcatccagcacttcgacctgaagatcttgtacgaactcaaaccgtaccttacgacggagtgctgccgttaccacacaatcctcaagagtgaatggggcacactcacccggatcccaaaggaagccgccatagctgaaggagccacctcgtttaaatttgttcatgtgtatccggggacagagtccccgtcccgttttaggtgagggtcacaactttaacagcaccggtgttgtccacaaagctcagggcattggcattggcagagtcaacgaagaaggcgttcttcttgccacccaagtctgcaagcacagccggagccaactccatgaactgcttcacggaagcaaccgaagtgctaagcgaactgacaccactgttcgtcgaggaaagccccgtggacaaagaggcgacagaactggttgccgtactgagtccagtcgaagcagacgtggacagagacgtagtggtgctcagtgctgtggacagggcagtggatagcgaaccaatcccactggatactgtagtggacagggatgccaccgaactgacggcagacgacagaccagtctgcgtcgaggtggacaacgattcaaccgaactgttggtcgaggacaaaccagtcgaggtagctgtggaaagcgacgagatacccgtagctgtgctggtagacaggctaacgatgtcttgggttgccttatcggcaattacacgagtctcttgaagaagagccacgtaaccagaaaggtcgaagatattgcgccaagcactggcaggctcatccgtgtatttccactgaagcatgtacccggcactgattcgcatctcgggactctgccccgtgatatccaccagaggaaccagcacctgccaattacccttggttccatcttggttcaggtatcgccaatcgagggtacggttgaccgtattctcgatcagctcgatatctttcggacgcaactcacccagatgctcggacacgtaggtgagcttctgaagattcttgtaaacctcacggacgattgggaatgcattgcccagaccaagatctaccatggccgacttgttctcgtcgtcttgaaatgcattacgcatcagacccacccccctttgttgaaacggatgttcgtagtcgacgaggaagtggacaccagatccttgtcgatcacgtcttggcagatagcctcgtacatcgcaatgtgactctgtgcgatgccttgggcttcctgcgtattcttgttactgtacagcttgtgggctacgtaggccaacagtgcttcctcaagcacgtcggggatctcgatctcttgttcggtctcgtcccgatcccagtagaggatagggtgcttcgcctgatacacgaggctcagtgcctgaccggataccggacggggaacctgaagcagtgcaccttgcggggagaataccgattctggttgagtgtcatcattcaacgggatctgatcaccgaagttgttgtacaccgacaggattcggatgatgtcatccgtgaacggttcacgggtcaagtccttgatgtacaggaagtcctgaccactcgtctcccactggctctcggcaaacttcgacttgagatggtagttcgtgatgtggtcgtggagttcgatcaccaagttgctttccttcagtgcgaaccgggagtacagacgaagcagtgcatcgttcaagtgacgaacgactttcggcttgaagtcatcccggagtaccccagaaccttcatcagcttcataaagattctggagttcaccgaaggagagcttttcgtaaagctcggcaagggtcattggttcctcacacaatataggaagaaagtcctgtagcttcctcgggttctgcatcgtcgtcgtacacgtccggtgcactgtgtgcacctaccggtgccgactccgatggcttccacggtttgaagtaaccaagcatggatatggtgtcgagtgcatcatccttacccttcaggccagactgcgtggccaatcggatctcggtcatgaactcggccatgatcttgctggtcttcatctcttcaggccagtagaacttgcctgccttgaaccaaggaaccacgaggttgaatcgggacagcttgtcgacgacaggtcggatacccggagcaccggacttctcggaggatgcaaagttgaaccagatattccggttcatcatctcagactggagccaagggataaatccaccctgctgacctgttacttcaatcccaaccatctgaggtttgtagatagagaccaatcggaagagatcatccacgttcttgtccatggtctgacgttcgcataccccatctacccagaaccaatcgccgttcgcattataggcccagaccgagatgacggagaagtcggcagtctgcttggccgacgtggcgaagtcagtcgtgatgtagaagttgaagttgctacggtacttgagcaagtcagcacgtttgtaccacttgatctctgcatcctgtacaagacgttcttcttccgacgtaatccggagcataagctcctgcatgaatgcagcaaccttaccggtcttcaccgatcgttcgtatgcgtcgagaacgtagtcgtagctgaaccgatcttcccaagctcccacgaactcttcgcgggtacaggggaaacgttcgcataccggccagacgttgacatcccaagcaccagattcaacggcttcaatgaggatgtcgtctttgttaaagggcgtcccattgaacacgactttccgtcgagtcggatcgagtgcatagtcaatgcctttgtaaacagtgtccttgatagcggccatgctggtcttggacttggcatcatcgtcgctgaccaagtcatcgagaatacagaagacaggtcgcttaccgaagatcttggaaccacggataccggtcttggcaccgaacatcttgaccccaagcttcaggccatcgacgttggtgaactccaagtagttgtccgtgaacttggcatcgggaatccacttctgaaggaattctgagttggcgtatcgatactcgatgttcttacgggcagacttcaccccgttgtccatggagtccgagacgtagatcatggcgtcgatacggccgaagttcggcaggtagccgaagactgcgatgtacagagtcatatactccatgaacagagtcgtcttagcagcacctcggaaacagaggttagccagatacgcactgtctccggagccgatcttgtccagcatcttcaggtggacagggggagtcttgttcgactcaccttccttaccattgaccagcttgatgaagttcatgaacgacaaagcgaactcggacggcatgtacgtatccgagttcagctctgcgtagttcacctgatccagccactcatcgagggactgggcgatcaatgttcgctttgttttcgggggttggctcatacgtcttcagcatccacgatcttctgagcagccagactcttcgctgtgaccccagcagcaatgaggtcacgttgctgttgagccaagctaccaagagttgccttcaggtctgcgagaccggacgactctcgcatgtcgatgttcaccagcgggcctgcttccttgggcttggccaagtgggacagcagactgttggcagcttccatgcgtaccttctccgactgtgccgatcgcatcagttgggcttggacgttgatggcttcctgatgcagatgctgattcagtacccacgaaggaaccagactcttctcgatgatcgagttgaccagcttacccttggcatatgcagacacataagcactgatgtccttggacttagcaccacgtgccagcagtgcagcatatcgctgaggaaaggtcttgaagtaggcatcttggttcgtgtcacccatcagcttgaagctgacgtacttcactgccgagacgtagtcttccgtcttgtacttcccctcagccatcacactggagtagctgacgaagttatcagcaatctgttgagccagcaccggatcactgacagcattattgatgtcgtcggcaatcgcttgcgtatccacgttacgcagtccggtcgggagtgccttggctacgagttctttggtcagcatagttgttctctcttacaggttcatctcagtaacacgcaggttcagccaatacgcataatcctgcattgcttcacgttgtttcaccatgtgacggtgttttctgggttcgagcgtatccacgaacgtcttgttggccagagctttattcagaccggtaagtcgttcctcaacctgccgtagttcgacgaccatgcgtgccttgtagtctttcggctcgttgaaactttggtaggccagaaggaacccaaagaacttccagcactggctgaatgcgtcttcataggcgtaacgcatgcccagttgagcattgaagttctcgacgaatacacacgaggactctcctcggatagtgaagcgatcgaagaacttgatcgaacagatggtcgtcttaccatcgtgaagcacatcgaagctcacgtcttgcatggcttcgagcatcatcagttcagttactttcggtgcagtcatggtcttctccttggaaaatgggatatactttaggctgtgtttagtcgtcagtcaaactctatctgtaatgggatgggaggcagagaactatggtgttccacacgtccccctacaaatatagtttgcaaattcacgagagcacgcttacactgaccctgttgttgcggttggtgagaagcgtgagcttgccatgagaccccagctagtggtggggataataactactagcagtcagagccaagaccaagaagtaactcctacttgtgtgatctgaccagtccccttgctgcctcaaggtgtattccctgatagctcagtaggtagagcacttgactgttaatcaaggtgtcgcaggttcgagccctgctcggggagccaaagaaagaaggattggatatctagtctttccctctttggtaggtatagatcaagaagagatcaccggcctatagccggagaccgttggtgagatcccaacacctctgccaaacagaaatgggaagacgttccctaaagtcctcggacacaatagccgtagtacacgagagtgtggcgaaaagacggggcaactattcaacagaccaccttagggtggtcttttcatttcaggagccaagatgctagatctcacagccccatggtacaaggatgacagcagcaactcaggcatctgggttcctgaatccttgggccatctgcttgatgtaacgaacaccctatatacctcgtaccacttctactgtactgaccgtgacacgtatgaagttctcaaagccctccttctcgtacaaggcttggctcataaggttgagtacggtaagccagacctgatcccacgtaaatgtgtagatacgtatggcatcttgcacatggccgcatgccagtggcaaccggaagatggatcccttcacaacaacccacgcttcgacgtgaaattcgcaaaggttagccatgactacatggacatctgatcgagtccgcagtgcaggaggatgaatgagtatccatgaccagtggtacatctctggtggggacatccaaccgatcagtatccaagaaggactggagaacgaacacaccttcatcgtcagtaacaaggacgaagaggctgaactccgtgcctatgcaatgttgctcggtcacactgctctcgtcaaagttatgctgggagcaagacagtacacccctgacttgaagcataaggatggtgtgttcaccttctacttaacccagtacagaggaagagatgacagattcaacttcatccccctataagttcgaccgatgggtagggtactgggcaggcgaggtacgtaacaataacgacttcgccttatcagccctgactacatccaaccctgacggtgacgtattcgtagtacggaaccaagaagacaaggaagagtgcctagccttcttcgccctgatgggctttaaccgtagggtgatcctctacttagaagacttcccccaaggtactgcacgagcattcataccttgtgcagatatgccaacccataaaccaagaccgtaccatccatgaccccaatcctcgaatgtaagtggaaggtagcgtctggtggcactacgctatttaaactatgtgaccttatgcgagttaaggacatagtctttgtcgtactgcacgaaggtgaccgcaacacgcttaaagcgtatctcttagttgagggatgtacgaacccagtcgtggatagctacgacctcgtacctatgaataagatgcccatggctcccatgcactagggtattagaactatctttgtataggatgcgattctggctaaaattttgcaagggcagaattttgatagctatgttccacagtagggtat